SEQUENCE LISTING (36 sequences)

SEQ ID NO: 1 - DNA, 2295 bp, Homo sapiens
aaagtctgcctctctagctacaacatttcttcaagaaaagaaagcagaagcccagaatcataatcgtgtccctgatgtaaaggcattaatggagagtcccgagggacagttatctctggagcccaaatctgatagtcacttccaagcatcacacactgggtgccagagccctttatgttcagcccagcgtcacactcctcagagccccttcaccaatcatgctgcagctgctggcaggaagactcttcgcagctgcatggggctggagtggtttccagagctctatcctggttaccttggactaggggtgttgccagggaagcctcagtgttggaatgcaatgacccagaagccacaacttatcagtccccagggggaaagactctcgcaagtttctttgttggaacgaagctcaactcatataaggagtttggaaccccctaccggacttacaacctccaacttctctctaatgaggctcttgggagctgtacaaaaaggctggatcaggtgcaacaccaccataaggaagagtggattcggtggcatcacgatgctcttcacaggatacttcgtcctgtgttgtagctggagtttcagacgtctgaaaaaattgtgccgacccctgccctggaagagcacagtacctccatgcattggtgtggcgaagacgactggggattgccgctctaaaacatgtttggattaggaagcacgtttaagtaggagaagccttcgtgacttctctctagtgccttcgtgccctgtgttgcccactgaattgccctgtaacacctaagtgtagtggtagcattaagggatagcttttcagccctcaaggttatcaggagcatttgtatcactgctataaataaagtagtatcacttgtcataatcagggtgctgaaacataatgaaaaggtttaacttagtcacagtaaatattttatatccctgaaaataagttatttaatgagaatagaactatttaatgatccatgagtcctaaaaggatatctgccttgtattgccaagtttaaggcaaaggtgagatcctgtattaataagaagacagcttttttgtttgacattggtaagtaccaagaattctaactcacatatagtaggaaacaacaaaaagtctccactgacttctttgaattagatactgagaatccacaatacagtagtgtggataactttcaagaagccattggaaacatctccacattctgggatgacttgtaaatgtctacagagtaatggaaggaaaggcttccttgtctgctcattgctgccacctgggttgggaaaataattgaagatgttttctcaagtccttcaagaatctcttcttaatgttcagaatagtttaagagcgttgttgaggtaaagtgatagctgattattttctgagatttagatctaaacaaactcagcatgacgaggaagcacacttcacagctccagggtgcaatactgatgtaaagttgcctcgtgaggatacatttttcgctcatgtatgcaaaattcctatcagttcatttgcacagccagttccaatcatgtatgtgatcaccagattaaaagcttcatccagaaagacaagactcctcagaacgaatagtagacaagtaagactgctacccacagaagccagtacgcttcaggtgtgaggcggggtaactactcatctcttcacagagccaaggaaagaaaaagaacgtctctaacatgatttaccattttaaacagccttataagttttgtctcttaaaatcatgctgcagaaggagggggtaaaagtcgtgttctgccctgtctgtggcattggagtgccccgctaggtaagaagcaatgcttagatcttgcttccaggcagcagcttgaattcccgaatcttcctgcaaggtgcatacaaatgcagcgtgagaatccatacacgtaatccatattcaccttcccatccatcccgcagaagaggcatggtgacacccaggctactgtccatgcttgagaggacgtatttgaaggttctgttactacaagttgggaatattcacggacatgcctgaataccccggctgtaactcacacgtggtctgtgtaagtggattaacctcggggcggccttgtttaatcctgaataatatctgaaagactgagtttacttgggaatgtgtagtttttctaatgcacattaaaatttattttagctgttaaaaataaaatcattttattatcaaaaaaaaaaaaaaaaaaaaaaaaaaaaaaaaaaaaaaaaaaaaaaaaaaa

SEQ ID NO: 2 - protein, 206 aa, Homo sapiens [sequence omitted]

SEQ ID NO: 3 - DNA, 16 bp, Artificial Sequence
  H-T11A Primer
aagctttttttttttc

SEQ ID NO: 4 - DNA, 13 bp, Artificial Sequence
  H-AP22 primer
aagcttttgatcc

SEQ ID NO: 5 - DNA, 3737 bp, Homo sapiens
cggcagctggaggtgtaatagtgcgggtagtgggtttggagaagttccgaggcggcggtggcgccggtcaggacaaggatagcggaaccgggccctgggcttgtcgctcaccatgccgacagtagaggagctttaccgcaattatggcatcctggccgatgccacggagcaagtgggccagcataaagatgcctatcaagtgatattggatggtgtgaaaggtggtactaaggaaaagcgattagcagctcaatttattccgaaattctttaagcattttccagaattggctgattctgctatcaatgcacagttagacctctgtgaggatgaagatgtatctattcgacgtcaagcaattaaagaactgcctcaatttgccactggagaaaatcttcctcgagtggcagatatactaacgcaacttttgcagacagatgactctgcagaatttaacctagtgaacaatgccctattaagtatatttaaaatggatgcaaaagggactttaggtgggttgttcagccaaatacttcaaggagaggacattgttagagaacgagcaattaaattcctttctacaaaacttaagactttaccagatgaagtcttaacaaaggaagtggaagagcttatactaactgaatccaaaaaggtcctagaagatgtgactggtgaagaatttgttctatttatgaagatactgtctgggttaaaaagcttacagacagtgagtggaagacagcaacttgtagagttggtggctgaacaggccgacctagaacagaccttcaatccctcggatcctgactgtgtggacaggctcttacagtgcactcggcaggcagtacccctcttctctaaaaatgtccattccacaaggtttgtgacatatttctgtgagcaggttctccctaacctcggtaccttgactaccccagtggaaggtcttgatatacagttggaggtattgaaattgttggcggagatgagttcattttgtggtgacatggaaaaactagaaacaaatttaaggaaactatttgataagttattggaatacatgcccctccctccagaagaggcagaaaatggagagaatgctggtaatgaagaacccaagctacagttcagttatgtggaatgtttgttgtacagttttcaccagttgggccgaaaacttccagatttcttaacagccaaactgaatgcagaaaagctcaaagatttcaaaatcaggctgcagtactttgcacggggcctgcaagtttatatcagacaacttcgcttagctctccagggtaaaacgggtgaggccttaaaaacagaagagaacaagattaaagtcgttgcattgaaaataacaaacaatatcaatgttttaatcaaggatctcttccacattcctccttcttataagagcacagtaacactatcctggaaacctgtacaaaaggttgagattgggcaaaagagagccagtgaagatacaacttcaggttcaccacccaagaaatcttcagcaggaccaaaaagagatgccaggcagatttataaccctcccagtgggaaatatagcagcaatttgggcaactttaattatgagaggagccttcaggggaagtagaggtggccgaggttggggcacacgaggaaatcgtagtcggggaagactctactgaataagacatcagcattcttcagcattgtcatgagcttaatatacttaaattctactactcattggattgccggggatgtccctttaaacagactgctgccttcagctaaaaacttaatgttctttatacctttgtatgtatgacctacttttgtaacagaccatggttgtgtccaaggtaaaaccacagtgatatttttggatgctttgtctgcaatcttgacttgtttttgcagtatcattattcagacttcaaattgtgaatcttttaaacatcttgataatttgttgttgagagctgttcattctaaaatgtaatgaaattcagtctagttctgctgataaagatcatcagttttgaaaggttactgattttcctcttccctcttagttttttacccaatatatggagaagagtaatggtcaatcttaacattttgttttaattgtttaataaagctgctgggcagtggtgcagcattcctacctagtgtcataaaagcaaaatacttacatagctttcttaaaatataggaatgacattacatttttaggagaaagtaagttgctttgcaccgcctacttaattcttttccatatattgtgatacaaacttttgaatatggaatcttactatttgaatagaaatgtgtatgtataatatacatacatacataagcatatatgtgtgtgtgtgtgtgtatatatatatatatgcatgctgtgaaacttgactacacaacataaatcactttttaaattccaggaacgggtagtctgacacggtgattatccttttgaggctgaatccgttattaacttgttatttaggtttttactcccagtagcaagggattctaagttagttgcacttacatgattattgttatttaaaactaagaataaaggctgcattttcaaagataaattggaattgctgttggtgaaataacaaccaaaatactgaatctgatgtacatacaggtttctacaggaagagatggtataatttacaatttggagatttaataaccagggctacccagaaaaagtgacttgataacatggtaccaataagtaagggatgctctctcggtttgcttttgccactttcaagattttaacttctcaggttattaatcaaaattattgtataagttagccaatagaatttttaggttaaaacaacagatggggggtttgtggagtgtttaatgtcatgggcatttttagtagcatagaccctttgttctgcatttgaatgtttcgtatatttttgtttcacagttaatcttccctccccaagtttgctattcaaatcaactgcctgaatgacatttctagtagtctgatgtatttttctgaggaatagtttgtgattccaatgcaggtgtcttcattaccattacctctacactgcagaagaagcaaaactcctttattagaattactgcacatgtgtatggggaaaatagttctgaaaggctagaatgatacaagtgagcaaaagttggtcagcttggctatggagtggtggcaataatctctaaacattccaaaagaccatgagctgaacctaaactcccttggaatctgaacaaaggaatataaaattgccatttgaaaactgaccagctaatctggacctcagagatagatcagccagtggcccaaagccatttcaagtacagaaattatagagactacagctaaataaatttgaacattaaatataattttaccactttttgtctttataagcatatttgtaaactcagaactgagcagaagtgactttactttctcaagtttgatactgagttgactgttcccttatccctcacccttccccttccctttcctaaggcaatagtgcacaacttaggttatttttgcttccgaatttgaatgaaaaacttaatgccatggatttttttcttttgcaagacacctgtttatcatcttgtttaaatgtaaatgtccccttatgcttttgaaataaatttccttttgtaattttaaaaaaaaaaaaaaaaaaaaaaaaaaaaaaaaaaaa

SEQ ID NO: 6 - protein, 504 aa, Homo sapiens
MPTVEELYRNYGILADATEQVGQHKDAYQVILDGVKGGTKEKRLAAQFIPKFFKHFPELADSAINAQLDLCEDEDVSIRRQAIKELPQFATGENLPRVADILTQLLQTDDSAEFNLVNNALLSIFKMDAKGTLGGLFSQILQGEDIVRERAIKFLSTKLKTLPDEVLTKEVEELILTESKKVLEDVTGEEFVLFMKILSGLKSLQTVSGRQQLVELVAEQADLEQTFNPSDPDCVDRLLQCTRQAVPLFSKNVHSTRFVTYFCEQVLPNLGTLTTPVEGLDIQLEVLKLLAEMSSFCGDMEKLETNLRKLFDKLLEYMPLPPEEAENGENAGNEEPKLQFSYVECLLYSFHQLGRKLPDFLTAKLNAEKLKDFKIRLQYFARGLQVYIRQLRLALQGKTGEALKTEENKIKVVALKITNNINVLIKDLFHIPPSYKSTVTLSWKPVQKVEIGQKRASEDTTSGSPPKKSSAGPKRDARQIYNPPSGKYSSNLGNFNYERSLQGK

SEQ ID NO: 7 - DNA, 16 bp, Artificial Sequence
  H-T11C primer
aagctttttttttttc

SEQ ID NO: 8 - DNA, 13 bp, Artificial Sequence
  H-AP23 primer
aagcttggctatg

SEQ ID NO: 9 - DNA, 1321 bp, Homo sapiens
tctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaact

SEQ ID NO: 10 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 11 - DNA, 16 bp, Artificial Sequence
  H-T11C primer
aagctttttttttttc

SEQ ID NO: 12 - DNA, 13 bp, Artificial Sequence
  H-AP23 primer
aagcttggctatg

SEQ ID NO: 13 - DNA, 1019 bp, Homo sapiens
ctataggcgcatggaaggttccctggaacgggaggcgccagcgggggcgctggccgccgtgctaaagcacagctcgacgttgccgcccgaaagcacccaggtccggggctacgacttcaaccgcggtgtgaattaccgcgcactgctggaggccttcggcaccaccggcttccaagcaaccaacttcgggcgcgctgtacagcaagtcaatgccatgatcgagaagaagctggaaccactgtcacaggatgaagaccagcacgcggacctgacccagagccgccgcccacttaccagctgcaccattttcctgggatatacatccaacctcatcagttcaggcatccgtgagaccattcgctaccttgtgcagcacaacatggtggacgtattggtgaccacagctggcggcgtggaggaagacctcatcaagtgcctggcgcccacatacttgggcgagtttagcctcagggggaaggagctccgggagaacgggatcaataggatcggaaacctgctggtgcccaatgagaattactgcaagtttgaggactggctgatgcccattctggaccagatggtgatggagcagaacacagagggtgtaaagtggacgccttctaagatgatcgcccggctgggcaaggagatcaacaacccagagtccgtgtattactgggcccagaagaaccacatccctgtgtttagtcccgcacttacagacggctcgctgggcgacatgatcttcttccattcctacaagaacccgggcctggtcctggacatcgttgaggcggaacggggccgactacgctgtttacatcaacacagcccaggagtttgatggctctgactcaggtgcccgaccagacgaggctgtctcctggggcaagatccgggtggatgcacagcccgtcaaggtctatgctgacgcctccctggtcttccccctgcttgtggctgaaacctttgcccagaagatggatgccttcatgcatgagaagaacgaggactgagcg

SEQ ID NO: 14 - protein, 277 aa, Homo sapiens
MEGSLEREAPAGALAAVLKHSSTLPPESTQVRGYDFNRGVNYRALLEAFGTTGFQATNFGRAVQQVNAMIEKKLEPLSQDEDQHADLTQSRRPLTSCTIFLGYTSNLISSGIRETIRYLVQHNMVDVLVTTAGGVEEDLIKCLAPTYLGEFSLRGKELRENGINRIGNLLVPNENYCKFEDWLMPILDQMVMEQNTEGVKWTPSKMIARLGKEINNPESVYYWAQKNHIPVFSPALTDGSLGDMIFFHSYKNPGLVLDIVEAERGRLRCLHQHSPGV

SEQ ID NO: 15 - DNA, 16 bp, Artificial Sequence
  H-T11C primer
aagctttttttttttc

SEQ ID NO: 16 - DNA, 13 bp, Artificial Sequence
  H-AP21 primer
aagctttctctgg

SEQ ID NO: 17 - DNA, 1142 bp, Homo sapiens
gagacccccaggttcaaaataagcctgtttggaacaacctcaggttttggaaccagtgggaccagcatgtttggcagtgcaactacagacaatcacaatcccatgaaggatattgaagtaacatcatctcctgatgatagcattggttgtctgtcttttagcccaccaaccttgccggggaactttcttattgcagggtcatgggctaatgatgttcgctgctgggaagttcaagacagtggacagaccattccaaaagcccagcagatgcacactgggcctgtgcttgatgtctgctggagtgacgatgggagcaaagtgtttacggcatcgtgtgataaaactgccaaaatgtgggacctcagcagtaaccaaacgatacagatcgcacagcatgatgctcctgttaaaaccatccattggatcaaagctccaaactacagctgtgtgatgactgggagctgggataagactttaaagttttgggatactcgatcgtcaaatcctatgatggttttgcaactccctgaaaggtgttactgtgctgacgtgatataccccatggctgtggtggcaactgcagagaggggcctgattgtctatcagctagagaatcaaccttctgaattcaggaggatagaatctccactgaaacatcagcatcggtgtgtggctatttttaaagacaaacagaacaagcctactggttttgccctgggaagtatcgaggggagagttgctattcactatatcaaccccccgaaccccgccaaagataacttcacctttaaatgtcatcgatctaatggaaccaacacttcagctcctcaggacatttatgcggtaaatggaatcgcgttccatcctgttcatggcacccttgcaactgtgggatctgatggtagattcagcttctgggacaaagatgccagaacaaaactaaaaacttcggaacagttagatcagcccatctcagcttgctgtttcaatcacaatggaaacatatttgcatacgcttccagctacgactggtcaaagggacatgaattttataatccccagaaaaaaaattacattttcctgcgtaatgcagccgaagagctaaagcccaggaataagaagtagtggctggagactctggc

SEQ ID NO: 18 - protein, 352 aa, Homo sapiens [sequence omitted]

SEQ ID NO: 19 - DNA, 16 bp, Artificial Sequence
  H-T11A primer
aagcttttttttttta

SEQ ID NO: 20 - DNA, 13 bp, Artificial Sequence
  H-AP21 primer
aagctttctctgg

SEQ ID NO: 21 - DNA, 3633 bp, Homo sapiens
cgcaatttccactcgcggggagcagcagcagcagaggcagcagcgggcggcgctgagccgccgccgccgccactgaggaagaagccggcccagccgccgccgcgtccggaccctcgcgcctggatcccagcgccccgatcccggcgccccaacccccacgcccgcctccgccaactttcacgctgcctcggcggcccggcccggctcgacgccaatggtggaggccatagtggagtttgactaccaggcccagcacgatgatgagctgacgatcagcgtgggtgaaatcatcaccaacatcaggaaggaggatggaggctggtgggagggacagatcaacggcaggagaggtttgttccctgacaactttgtaagagaaataaagaaagagatgaagaaagaccctctcaccaacaaagctccagaaaagcccctgcacgaagtgcccagtggaaactctttgctgtcttctgaaacgattttaagaaccaataagagaggcgagcgacggaggcgccggtgccaggtggcattcagctacctgccccagaatgacgatgaacttgagctgaaagttggcgacatcatagaggtggtaggagaggtagaggaaggatggtgggaaggtgttctcaacgggaagactggaatgtttccttccaacttcatcaaggagctgtcaggggagtcggatgagcttggcatttcccaggatgagcagctatccaagtcaagtttaagggaaaccacaggctccgagagtgatgggggtgactcaagcagcaccaagtctgaaggtgccaacgggacagtggcaactgcagcaatccagcccaagaaagttaagggagtgggctttggagacattttcaaagacaagccaatcaaactaagaccaaggtcaattgaagtagaaaatgactttctgccggtagaaaagactattgggaagaagttacctgcaactacagcaactccagactcatcaaaaacagaaatggacagcaggacaaagagcaaggattactgcaaagtaatatttccatatgaggcacagaatgatgatgaattgacaatcaaagaaggagatatagtcactctcatcaataaggactgcatcgacgtaggctggtgggaaggagagctgaacggcagacgaggcgtgttccccgataacttcgtgaagttacttccaccggactttgaaaaggaagggaatagacccaagaagccaccgcctccatccgctcctgtcatcaaacaaggggcaggcaccactgagagaaaacatgaaattaaaaagatacctcctgaaagaccagaaatgcttccaaacagaacagaagaaaaagaaagaccagagagagagccaaaactggatttacagaagccctccgttcctgccataccgccaaaaaagcctcggccacctaagaccaattctctcagcagacctggcgcactgcccccgagaaggccggagagaccggtgggtccgctgacacacaccaggggtgacagtccaaagattgacttggccggcagttcgctatctggcatcctggacaaagatctctcggaccgcagcaatgacattgacttagaaggttttgactccgtggtatcatctactgagaaactcagtcatccgaccacaagcagaccaaaagctacagggaggcggcctccgtcccagtccctcacatcttcatccctttcaagccctgatatcttcgactccccaagtcccgaagaggataaggaggaacacatttcacttgcgcacagaggagtggacgcgtcaaagaaaacttccaagactgttaccatatcccaagtgtctgacaacaaagcatccctgccgcccaagccggggaccatggcagcaggtggcggtgggccagcccctctgtcctcagcggcgccctcccccctgtcatcctctttgggaacagctggacacagagccaactccccgtctctgttcggcacggaaggaaaaccaaagatggagcctgcggccagcagccaggcggccgtggaggagctaaggacacaggtccgcgagctgaggagcatcatcgagaccatgaaggaccagcagaaacgagagattaaacagttattgtctgagttggatgaagagaagaaaatccggcttcggttgcagatggaagtgaacgacataaagaaagctctacaatcaaaatgaatacttgatcaatgaaatgtcacattattcatcctgagtccgagactcaaattttctgccccagccaaaataatcttgtgccaaaagattaaaggtttgcctcaaaatgtccctgtttgaaagattagcacaaaagtcttgatagcacaacacaaattccatccaagaggagaatcttccccagggtttagtcctggggctggcactcgttgtgacttacacagagcaaaattgtgctaaaggcttttctactctgagatctcaatgcgaaatgaaaactcaggcagtttagtccatagtggtactattttgatgatattttccattaataaaatgtaatttcagattattcgtttacaagctttataattttatgattttttaatcgtgttttgtcacagacttccctagtgtttgtactacacgtagtcagaagcgagtgtccttttcttttgcttcaggctaagagctgcctcgctctttgtccccccattaggattctattacatatgcaattgtaggttcaacctgtccctttccctgccagcaaaccccaccaccctaagagaaattttagcttatatatgacggtatatttacaaaaagagaaagagaaaatctggtatttgcaatgatctgtgccttctttttaccaccctcttgattggagcttttgtgatgcagctaccatgattcaaaaaaattaaaaattaaaaaaaaaaaatctgccacttatccaagtccactagaggccactgtcttcaaagcttctctcaccctagccaaaggtcctaagaggagacagctgtgaagttgggcgtgctctgtggtaccagctgtgacttttctatttctcctagttttaggttgttcatgaaactagaaatgtcatcctgcttgatttttcatcagccaagttaaacccctgctttctgtcctttgcaccttttgcgtgaacagaatatgcattattaaagcaaaaataaataaaagtaaaatgcaaatgaaaaaaaaaaaaaaaaaaaaaaaaaaaaaaaaaaggccgcccccaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaataaaaaaaaaaaaaaaaaaaaaaaaaaaaaaaaaaaaaaaaaaaaaaaaaaaa

SEQ ID NO: 22 - protein, 665 aa, Homo sapiens
MVEAIVEFDYQAQHDDELTISVGEIITNIRKEDGGWWEGQINGRRGLFPDNFVREIKKEMKKDPLTNKAPEKPLHEVPSGNSLLSSETILRTNKRGERRRRRCQVAFSYLPQNDDELELKVGDIIEVVGEVEEGWWEGVLNGKTGMFPSNFIKELSGESDELGISQDEQLSKSSLRETTGSESDGGDSSSTKSEGANGTVATAAIQPKKVKGVGFGDIFKDKPIKLRPRSIEVENDFLPVEKTIGKKLPATTATPDSSKTEMDSRTKSKDYCKVIFPYEAQNDDELTIKEGDIVTLINKDCIDVGWWEGELNGRRGVFPDNFVKLLPPDFEKEGNRPKKPPPPSAPVIKQGAGTTERKHEIKKIPPERPEMLPNRTEEKERPEREPKLDLQKPSVPAIPPKKPRPPKTNSLSRPGALPPRRPERPVGPLTHTRGDSPKIDLAGSSLSGILDKDLSDRSNDIDLEGFDSVVSSTEKLSHPTTSRPKATGRRPPSQSLTSSSLSSPDIFDSPSPEEDKEEHISLAHRGVDASKKTSKTVTISQVSDNKASLPPKPGTMAAGGGGPAPLSSAAPSPLSSSLGTAGHRANSPSLFGTEGKPKMEPAASSQAAVEELRTQVRELRSIIETMKDQQKREIKQLLSELDEEKKIRLRLQMEVNDIKKALQSK

SEQ ID NO: 23 - DNA, 16 bp, Artificial Sequence
  H-T11A primer
aagcttttttttttta

SEQ ID NO: 24 - DNA, 13 bp, Artificial Sequence
  H-AP36 primer
aagcttcgacgct

SEQ ID NO: 25 - DNA, 4639 bp, Homo sapiens
agcggacggtccttgcattggcctccggcaggcgccccccgggggcgggaagctgcctcacagcatggaaccacaggttactctaaatgtgacttttaaaaatgaaattcaaagctttctggtttctgatccagaaaatacaacttgggctgatatcgaagctatggtaaaagtttcatttgatctgaatactattcaaataaaatacctggatgaggaaaatgaagaggtatccatcaacagtcaaggagaatatgaagaagcgcttaagatggcagttaaacagggaaaccaactgcagatgcaagtccacgaagggcaccatgtcgttgatgaagccccacccccagttgtaggagcaaaacgactagctgccagggcagggaagaagccacttgcacattactcttcactggtgagagtcttgggatcagacatgaagaccccagaggatcctgcagtgcagtcgtttccacttgttccatgtgacacagaccagcctcaagacaagcccccagactggttcacaagctacctggagacgttcagagaacaagtggttaacgaaacggttgagaagcttgaacagaaattacatgaaaagcttgtcctccagaacccatccttgggttcttgtccctcagaagtctcaatgcctacttcagaagaaacattgtttttgccagaaaaccagttcagctggcatattgcttgcaacaactgccaaagaaggattgttggtgtccgctaccagtgtagcctatgcccatcctacaatatctgtgaagattgtgaagcagggccatatggccatgacactaaccacgtcctgctgaagttgcggagacctgttgtgggctcctctgaaccgttctgtcactcaaagtactctactcctcgtcttcctgctgctctggaacaagtcaggctccagaaacaggttgataagaactttcttaaagcagaaaagcaaaggttgcgagctgagaagaaacaacgtaaagcagaggtcaaggaacttaaaaagcagcttaaactccataggaaaattcacctgtggaattcaatccatggactccagagccccaagtctcctttaggccgacctgagagcttgctccagtctaataccctgatgctccctttgcagccctgtacctccgttatgccaatgctcagtgcagcatttgtggatgagaatttgcctgatgggactcaccttcagccaggaaccaagtttatcaaacactggaggatgaaaaatacaggaaatgtaaagtggagtgcagacacaaagctcaagttcatgtggggaaacctgactttggcttccacagaaaagaaggatgttttggttccctgcctcaaggccggccatgtgggagttgtatctgtggagttcattgccccagccttggagggaacgtatacttcccattggcgtctttctcacaaaggccagcaatttgggcctcgggtctggtgcagtatcatagtagatcctttcccctccgaagagagccctgataacattgaaaagggcatgatcagctcaagcaaaactgatgatctcacctgccagcaagaggaaacttttcttctggctaaagaagaaagacagcttggtgaagtgactgagcagacagaagggacagcagcctgcatcccacagaaggcaaaaaatgttgccagtgagagggagctctacatcccatctgtggatcttctgactgcccaggacctgctgtcctttgagctgttggatataaacattgttcaagagttggagagagtgccccacaacacccctgtggatgtgactccctgcatgtctcctctgccacatgacagtcctttaatagagaagccaggcttggggcagatagaggaagagaatgaaggggcaggatttaaagcacttcctgattctatggtgtcagtaaagaggaaggctgagaacattgcttctgtggaggaagcagaagaagacctgagtgggacccagtttgtgtgtgagacagtaatccgatcccttaccttggatgctgccccagaccacaaccctccttgcagacagaagtccttgcagatgacatttgccttgcctgaaggaccacttggaaatgagaaggaggagattatccatatcgctgaggaagaagctgtcatggaggaggaggaggatgaggaggatgaggaggaggaggatgagctcaaagatgaagttcaaagtcagtcctctgcttcctcagaggattacatcatcatcctgcctgagtgctttgataccagccgccccctgggggattctatgtacagctctgcgctctcacagccaggcctggagcgaggtgctgaaggcaagcctggggttgaggctgggcaggaaccagctgaggctggggaaagactccctggaggggagaaccagccacaggagcacagcataagtgacatcctcacgacctcacagactctggaaacagtgcccctaatcccagaggtagtggagcttccaccgtcactgcccaggagctctccttgtgtacatcatcatggttccccaggagtggatttaccagttaccataccagaagtttcttcagtccctgatcagatcagaggagagcccagaggctcatcaggacttgtaaacagcagacagaagagctatgaccactcaaggcaccatcatgggagcagcattgctggaggactggtgaagggggctttgtctgttgctgcctctgcatacaaggccctgtttgctgggccaccagtcactgcacagccaataatttctgaagatcagacagcagccctgatggcccatctctttgaaatgggattctgtgacaggcagctgaacctacggctgctgaagaaacacaattacaatatcctgcaggttgtgacagaacttcttcagttaaacaacaacgactggtacagccaacgctattgaggagtgaccttgtattaaataactgcctgctgctcagagatgatctttattctgtcattggggtatgggatagaagcccttgcttatttttaatctgatgaatctgtatagagcccatcgttgagttaccaagacaatacctgctacagtattttggggagcaaactaaagaccagaacttaaattttcactttagacattggatgaatagtatgaagacagtttttcagttgatttggataaaactattttagtgcattgacaagtgtaacttcaacttcatatagaaccatttttctttctgcttttattgaaactgagtatttttctttggctaatgtggattttttatggggatatctgttaattttcaggttttgaaagacattaacctcggaagttgtttttaagaattattctcataattcttattctcataatttctgtaatccacctcaagcttcatagttatttggcattgaaataacacccagagcatgatagaaatgttgttactcttcctctctcaaggagaaagtaattttcctgcaatacttaataattggcaccgttgctttctaaagactccatggtgcattcaagagtatccaacttcaagggaatctccgcatttcaatgaaaggaggaagagtgtgctgataaacctaccagcacctattgagcaatgtctattatagtaattttgcatacatttttatttaagggaaaaaatataggtattgtgaaatattttgctaatcttatagaaaaggaaaaaatcccgttatttaaagggaaaagtaaatttaacagttgccttttttcttaatgtcagggcagatcttattttacagtacagtgggggaaatagaaaacatgtgaaaggcaaaaggcaggctcctaaattaatgtcagtgaagttcagggtgggcaaatgagtgtgtgtgaggtataggaaatgctgatgacttctttaatgcttgaagtccgttcacaggtatctagccctagaatgcctagaacaggaagaggcagctggtgttctgcaaaacttggacaggggcaaagttgctgaaaaagttttggtttaacccgaagataagtggaaaagagcttgtccatgaacccaggttctcactctgtttacagaagtgtgttgagtacagttggtgaaggaagaggtaacaaaaaatgctaaatattttatccatgaaaatgacttccagaaaaggaagaatatgaaccccagaccgaaggggaaaagatagttaatagtattatctaacctggttggtatttgtaatgaatggtgattttaattagtcattagccataatgatgtttatttacagtataactcctgaatgctacttaaataaaccaggattcaaactgcaagccagccaggccgttcattatttaaaacgttttaatcggggcttccgggtagaaggtggagcggcagggtgtaattgggttgatgggtgggacctgtcttgaccatcggagttttataatcgagggccaggagggcccgggttgctctcctggttatgtatgtacttgtacataaccaccttaagaatggtgaaataaatgttcttggaaattcctaaaaaaaaaaaaaaaaaaaaaaaaaaaaaaaaaaaaaaaaaaaaaaaaaaaaaaaaaaaa

SEQ ID NO: 26 - protein, 966 aa, Homo sapiens
MEPQVTLNVTFKNEIQSFLVSDPENTTWADIEAMVKVSFDLNTIQIKYLDEENEEVSINSQGEYEEALKMAVKQGNQLQMQVHEGHHVVDEAPPPVVGAKRLAARAGKKPLAHYSSLVRVLGSDMKTPEDPAVQSFPLVPCDTDQPQDKPPDWFTSYLETFREQVVNETVEKLEQKLHEKLVLQNPSLGSCPSEVSMPTSEETLFLPENQFSWHIACNNCQRRIVGVRYQCSLCPSYNICEDCEAGPYGHDTNHVLLKLRRPVVGSSEPFCHSKYSTPRLPAALEQVRLQKQVDKNFLKAEKQRLRAEKKQRKAEVKELKKQLKLHRKIHLWNSIHGLQSPKSPLGRPESLLQSNTLMLPLQPCTSVMPMLSAAFVDENLPDGTHLQPGTKFIKHWRMKNTGNVKWSADTKLKFMWGNLTLASTEKKDVLVPCLKAGHVGVVSVEFIAPALEGTYTSHWRLSHKGQQFGPRVWCSIIVDPFPSEESPDNIEKGMISSSKTDDLTCQQEETFLLAKEERQLGEVTEQTEGTAACIPQKAKNVASERELYIPSVDLLTAQDLLSFELLDINIVQELERVPHNTPVDVTPCMSPLPHDSPLIEKPGLGQIEEENEGAGFKALPDSMVSVKRKAENIASVEEAEEDLSGTQFVCETVIRSLTLDAAPDHNPPCRQKSLQMTFALPEGPLGNEKEEIIHIAEEEAVMEEEEDEEDEEEEDELKDEVQSQSSASSEDYIIILPECFDTSRPLGDSMYSSALSQPGLERGAEGKPGVEAGQEPAEAGERLPGGENQPQEHSISDILTTSQTLETVPLIPEVVELPPSLPRSSPCVHHHGSPGVDLPVTIPEVSSVPDQIRGEPRGSSGLVNSRQKSYDHSRHHHGSSIAGGLVKGALSVAASAYKALFAGPPVTAQPIISEDQTAALMAHLFEMGFCDRQLNLRLLKKHNYNILQVVTELLQLNNNDWYSQRY

SEQ ID NO: 27 - DNA, 16 bp, Artificial Sequence
  H-T11A primer
aagcttttttttttta

SEQ ID NO: 28 - DNA, 13 bp, Artificial Sequence
  H-AP33 primer
aagcttgctgctc

SEQ ID NO: 29 - DNA, 833 bp, Homo sapiens
cggcttccagtccgcggagggcgaggcggcgtggacagcggccccggcacccagcgccccgccgcccgcaagccgcgcgcccgtccgccgcgccccgagcccgccgcttcctatctcagcgccctgccgccgccgccgcggcccagcgagcggccctgatgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaaatgtctcagcccctcgttcttggtcctgtcccttggaacctttgtacgctttgctcaatcaaaaacaaaaaaaaaaaaaaaaaaaaaaaaaaaaaa

SEQ ID NO: 30 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 31 - DNA, 16 bp, Artificial Sequence
  H-T11G primer
aagctttttttttttg

SEQ ID NO: 32 - DNA, 13 bp, Artificial Sequence
  H-AP26 primer
aagcttgccatgg

SEQ ID NO: 33 - DNA, 2364 bp, Homo sapiens
gcagaggaaagggtgaagggagtctgggcaagcaaagcatagagatggtggggtggtggtggggttgaagaaacttgttggtataattgtcataggacttgcctaaaatattattaaaattacgggagtgtactcagctttgagcctaggagaaaatgccactgtgtgcatccattttaaagggttccctcataaaaaaatgttattccccattatcacatcagtacactgctttgaaaacaaaacttttcaacatgggcatactgggctacatggaaaatgacatcacccaggagtgatttctctttatatatattatttctgcagttaccatccttatctgagttatcacagttcatgaatctaagaggcggaactctacatcattagtaagaggttccaccaaagtctaaagttgtattcacttgtgtttgatgaactatctttaaaagaccataggtctatcattatttcttagacataatctaaagaaaaacagactagagaagccacctggttgtaacagaataagcagaagtttacagcatgatagtccaagtggtgataactttaaataaaactcaaatttttactgtttgtagacaggaatgctgtcctagagaacctcctcctcaaccagctacgtacatagttttatcctatgcattcctgttttctgtgttttttgttttttttttttgagacagagtctcgctctgtcacccaggctggagtgcagtggtgcgacctcagctcactgaaacctctgcctcccgggttcaagcgattctcctgcatcagcctcccgagtagctaggattacaggcgcccgccactacgcccagctaatttgtggtatttttagtagagacagggtttcaccatgttggccaggctggtctcgaactcctgacctcatgatccgcccgccttgacctcccaaagtgctgggattacaggcatgagccaccgcacccagcctgcattcctgtttttttaatggttttggagggtagcagtagagatggggtctcactatgttgcccagtctagtcttgaactcctgggctacagttaccctcctacctcggcttcccaaagtgctcggattacaggtgtgagccactgtgcctagcctataatgatcattttaatgtttcccatgcactcatttagtttgaaccttcacagcaacccaatgaggtaatactcccatttcacatataatactgagagatgagttgcacaagattatacactgttaagtagcagagccagaatggacttcagaatcccaactacaatacaaatgtttatttaaataaagaagaaagctattgtacaaatatcactcttcaggtttagcttacagagccatggctatggattcttagctctgtaaggaagtgcttctataaattcttaggtttagagatgataccatctgggtacctttgcttgaaccgtgcaaccacatctgggtctagtaggtggatcccatccagttggtttccaagggtgatcctgaaacagtgtaaaaggaggggcaaaccagaaatcctggaattagagggtttaatattgttaaaaaatgcataccaaatgaagactgcctatcatcatatcaaatatgccaattctaaaaagagcttaacattagaatagtatatggtagaattactagttcagaattggcatagattctggtgttaaaatagactggatctgtattatctgagggttagtaactaatgcttagccaggcctgcttcacagagttgctaccagggagtattctttggataagcaaatgctagcagcatgtgttttaagctctgttaaggggtgaaagatgtaattattgacagattaaatagataacttcgtaaccaccagggggcagattcaatacatcacagaatggctgaggaagatccttgggttgtgaagagagtagaaaccctagggagcagtgcttttgggtcctagaacctgttgagtttctaatgaatatttgtagaatctcataaaacagtttaaatacaagcttaagtggcttatgaatcctgtgaagctcatttatggactagtgtaaaacaatgtgaagctctactaagttctgtccttaatcataaataatagccccttgaggactagcctgttctctggtcaccttaccagttgggttgcacattgtgtggtcgtccaaataactcaatcttgcgagtgccaggagatagtctttcaatcatgccatagatttcatctggtttatgactggtggaacgaacctaggaaataaaaactagctgctttttaagttaaaaaaaaaaaaaaaaaaaaaaaaaaaaaaaaaaaaaaaaa

SEQ ID NO: 34 - protein, 76 aa, Homo sapiens
MIPSGYLCLNRATTSGSSRWIPSSWFPRVILKQCKRRGKPEILELEGLILLKNAYQMKTAYHHIKYANSKKSLTLE

SEQ ID NO: 35 - DNA, 16 bp, Artificial Sequence
  H-T11G primer
aagctttttttttttg

SEQ ID NO: 36 - DNA, 13 bp, Artificial Sequence
  H-AP23 primer
aagcttggctatg

(19) United States
(12) Patent Application Publication
(10) Pub. No.: US 2008/0213764 A1
(43) Pub. Date: Sep. 4, 2008

(54) Human Protooncogene and Protein Encoded Therein

(76) Inventors: Hyun-Kee Kim, Seoul (KR); Jin-Woo Kim, Seoul (KR)

Correspondence Address: HAMRE, SCHUMANN, MUELLER & LARSON, P.C., P.O. BOX 2902, MINNEAPOLIS, MN, 55402-0902, US

(21) Appl. No.: 11/794,402

(22) PCT Filed: Dec. 28, 2005
(86) PCT No.: PCT/KR05/04617

§ 371 Date: Nov. 13, 2007

(30) Foreign Application Priority Data
Dec. 28, 2004 (KR) 10-2004-0114281

(51) Int. Cl. C12Q 1/68; C07K 14/00; C12N 15/11; C12N 15/00; C12Q 1/00

(52) U.S. Cl. 435/6; 530/350; 536/23.5; 435/320.1; 435/4

(57) ABSTRACT

Disclosed are a novel protooncogene and a protein encoded therein. The protooncogene of the present invention, which is a novel gene that takes part in human carcinogenesis and simultaneously has an ability to induce cancer metastasis, may be effectively used for diagnosing the cancers, including lung cancer, leukemia, uterine cancer, lymphoma, colon cancer, skin cancer, etc., as well as producing transformed animals, etc.

### TECHNICAL FIELD

The present invention relates to a novel protooncogene which has no homology with the protooncogenes reported previously, but has an ability to induce cancer metastasis; and a protein encoded therein.

### BACKGROUND ART

Generally, it has been known that the higher animals, including human, have approximately 30,000 genes, but only approximately 15% of the genes are expressed in each subject. Accordingly, it was found that all phenomena of life, namely generation, differentiation, homeostasis, responses to stimulus, control of cell cycle, aging and apoptosis (programmed cell death), etc. were determined depending on which genes are selected and expressed (Liang, P. and A. B. Pardee, Science 257: 967-971, 1992).

The pathological phenomena such as oncogenesis are induced by the genetic variation, resulting in changed expression of the genes. Accordingly, comparison of the gene expressions between different cells may be a basic and fundamental approach to understand various biological mechanisms. For example, the mRNA differential display method proposed by Liang and Pardee (Liang, P. and A. B. Pardee, Science 257: 967-971, 1992) has been effectively used for searching tumor suppressor genes, genes relevant to cell cycle regulation, and transcriptional regulatory genes relevant to apoptosis, etc., and also widely employed for specifying correlations of the various genes that rise only in one cell.

Putting together the various results of oncogenesis, it has been reported that various genetic changes such as loss of specific chromosomal heterozygosity, activation of the protooncogenes, and inactivation of other tumor suppressor genes including the p53 gene was accumulated in the tumor tissues to develop human tumors (Bishop, J. M., Cell 64: 235-248, 1991; Hunter, T., Cell 64: 249-270, 1991). Also, it was reported that 10 to 30% of the cancer was activated by amplifying the protooncogenes. As a result, the activation of protooncogenes plays an important role in the etiological studies of many cancers, and therefore there have been attempts to specify the role.

Accordingly, the present inventors found that a mechanism for generating lung cancer and cervical cancer was studied in a protooncogene level, and therefore the protooncogene, named a human migration-inducing gene, showed a specifically increased level of expression only in the cancer cell. The protooncogene may be effectively used for diagnosing, preventing and treating the various cancers such as lung cancer, leukemia, uterine cancer, lymphoma, colon cancer, skin cancer, etc.

### DISCLOSURE OF INVENTION

Accordingly, the present invention is designed to solve the problems of the prior art, and therefore it is an object of the present invention to provide novel protooncogenes and their fragments.

It is another object of the present invention to provide recombinant vectors containing each of the protooncogenes and their fragments; and microorganisms transformed by each of the recombinant vectors.

It is still another object of the present invention to provide proteins encoded by each of the protooncogenes; and their fragments.

It is still another object of the present invention to provide kits for diagnosing cancer and cancer metastasis, including each of the protooncogenes or their fragments.

It is yet another object of the present invention to provide kits for diagnosing cancer and cancer metastasis, including each of the proteins or their fragments.

In order to accomplish the above object, the present invention provides a protooncogene having a DNA sequence of SEQ ID NO: 1; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 2; or its fragments.

The present invention provides a protooncogene having a DNA sequence of SEQ ID NO: 5; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 6; or its fragments.

The present invention provides a protooncogene having a DNA sequence of SEQ ID NO: 9; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 10; or its fragments.

The present invention provides a protooncogene having a DNA sequence of SEQ ID NO: 13; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 14; or its fragments.

The present invention provides a protooncogene having a DNA sequence of SEQ ID NO: 17; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 18; or its fragments.

The present invention provides a protooncogene having a DNA sequence of SEQ ID NO: 21; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 22; or its fragments.

The present invention provides a protooncogene having a DNA sequence of SEQ ID NO: 25; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 26; or its fragments.

The present invention provides a protooncogene having a DNA sequence of SEQ ID NO: 29; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 30; or its fragments.

The present invention provides a protooncogene having a DNA sequence of SEQ ID NO: 33; or its fragments.

According to the another object, the present invention provides a recombinant vector containing the protooncogene or its fragments; and a microorganism transformed by the recombinant vector.

According to the still another object, the present invention provides a protein having an amino acid sequence of SEQ ID NO: 34; or its fragments.

According to the still another object, the present invention provides kits for diagnosing cancer and cancer metastasis including the protooncogenes and their fragments.

According to the still another object, the present invention provides kits for diagnosing cancer and cancer metastasis including the protooncoproteins and their fragments.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a gel diagram showing a result of the differential display reverse transcription-polymerase chain reaction (DDRT-PCR) to determine whether or not an L276811 DNA fragment is expressed in a normal lung tissue, a left lung cancer tissue, a metastatic lung cancer tissue metastasized from the left lung to the right lung, and an A549 lung cancer cell;

FIG. 2 is a gel diagram showing a result of the differential display reverse transcription-polymerase chain reaction (DDRT-PCR) to determine whether or not a CC231 DNA fragment is expressed in a normal exocervical tissue, a cervical tumor tissue, a metastatic lymph node tumor tissue and a CUMC-6 cancer cell;

FIG. 3 is a gel diagram showing a result of the differential display reverse transcription-polymerase chain reaction (DDRT-PCR) to determine whether or not an L789 DNA fragment is expressed in a normal lung tissue, a left lung cancer tissue, a metastatic lung cancer tissue metastasized from the left lung to the right lung, and an A549 lung cancer cell;

FIG. 4 is a gel diagram showing a result of the differential display reverse transcription-polymerase chain reaction (DDRT-PCR) to determine whether or not an L986 DNA fragment is expressed in a normal lung tissue, a left lung cancer tissue, a metastatic lung cancer tissue metastasized from the left lung to the right lung, and an A549 lung cancer cell;

FIG. 5 is a gel diagram showing a result of the differential display reverse transcription-polymerase chain reaction (DDRT-PCR) to determine whether or not an L1284 DNA fragment is expressed in a normal lung tissue, a left lung cancer tissue, a metastatic lung cancer tissue metastasized from the left lung to the right lung, and an A549 lung cancer cell;

FIG. 6 is a gel diagram showing a result of the differential display reverse transcription-polymerase chain reaction (DDRT-PCR) to determine whether or not a CA367 DNA fragment is expressed in a normal exocervical tissue, a cervical tumor tissue, a metastatic lymph node tumor tissue and a CUMC-6 cancer cell;

FIG. 7 is a gel diagram showing a result of the differential display reverse transcription-polymerase chain reaction (DDRT-PCR) to determine whether or not a CA335 DNA fragment is expressed in a normal exocervical tissue, a cervical tumor tissue, a metastatic lymph node tumor tissue and a CUMC-6 cancer cell;

FIG. 8 is a gel diagram showing a result of the differential display reverse transcription-polymerase chain reaction (DDRT-PCR) to determine whether or not a CG263 DNA fragment is expressed in a normal exocervical tissue, a cervical tumor tissue, a metastatic lymph node tumor tissue and a CUMC-6 cancer cell;

FIG. 9 is a gel diagram showing a result of the differential display reverse transcription-polymerase chain reaction (DDRT-PCR) to determine whether or not a CG233 DNA fragment is expressed in a normal exocervical tissue, a cervical tumor tissue, a metastatic lymph node tumor tissue and a CUMC-6 cancer cell.

FIG. 10(a) is a gel diagram showing a northern blotting result to determine whether or not the MIG3 protooncogene of the present invention is expressed in the normal lung tissue, the left lung cancer tissue, the metastatic lung cancer tissue metastasized from the left lung to the right lung, and the A549 and NCI-H358 lung cancer cell lines, and FIG. 10(b) is a diagram showing a northern blotting result obtained by hybridizing the same sample as in FIG. 10(a) with β-actin probe;

FIG. 11 is a gel diagram showing a northern blotting result to determine whether or not the MIG8 protooncogene of the present invention is expressed in the normal exocervical tissue, the uterine cancer tissue, the metastatic cervical lymph node tissue and the cervical cancer cell line;

FIG. 12 is a diagram showing a northern blotting result obtained by hybridizing the same sample as in FIG. 11 with β-actin probe;

FIG. 13(a) is a gel diagram showing a northern blotting result to determine whether or not the MIG10 protooncogene of the present invention is expressed in the normal lung tissue, the left lung cancer tissue, the metastatic lung cancer tissue metastasized from the left lung to the right lung, and the A549 and NCI-H358 lung cancer cell lines, and FIG. 13(b) is a diagram showing a northern blotting result obtained by hybridizing the same sample as in FIG. 13(a) with β-actin probe;

FIG. 14(a) is a gel diagram showing a northern blotting result to determine whether or not the MIG13 protooncogene of the present invention is expressed in the normal lung tissue, the left lung cancer tissue, the metastatic lung cancer tissue metastasized from the left lung to the right lung, and the A549 and NCI-H358 lung cancer cell lines, and FIG. 14(b) is a diagram showing a northern blotting result obtained by hybridizing the same sample as in FIG. 14(a) with β-actin probe;

FIG. 15(a) is a gel diagram showing a northern blotting result to determine whether or not the MIG14 protooncogene of the present invention is expressed in the normal lung tissue, the left lung cancer tissue, the metastatic lung cancer tissue metastasized from the left lung to the right lung, and the A549 and NCI-H358 lung cancer cell lines, and FIG. 15(b) is a diagram showing a northern blotting result obtained by hybridizing the same sample as in FIG. 15(a) with β-actin probe;

FIG. 16 is a gel diagram showing a northern blotting result to determine whether or not the MIG18 protooncogene of the present invention is expressed in the normal exocervical tissue, the uterine cancer tissue, the metastatic cervical lymph node tissue and the cervical cancer cell line;

FIG. 17 is a diagram showing a northern blotting result obtained by hybridizing the same sample as in FIG. 16 with β-actin probe;

FIG. 18 is a gel diagram showing a northern blotting result to determine whether or not the MIG19 protooncogene of the present invention is expressed in the normal exocervical tissue, the uterine cancer tissue, the metastatic cervical lymph node tissue and the cervical cancer cell line;

FIG. 19 is a diagram showing a northern blotting result obtained by hybridizing the same sample as in FIG. 18 with β-actin probe;

FIG. 20 is a gel diagram showing a northern blotting result to determine whether or not the MIG5 protooncogene of the present invention is expressed in the normal exocervical tissue, the uterine cancer tissue, the metastatic cervical lymph node tissue and the cervical cancer cell line;

FIG. 21 is a diagram showing a northern blotting result obtained by hybridizing the same sample as in FIG. 20 with β-actin probe;

FIG. 22 is a gel diagram showing a northern blotting result to determine whether or not the MIG7 protooncogene of the present invention is expressed in the normal exocervical tissue, the uterine cancer tissue, the metastatic cervical lymph node tissue and the cervical cancer cell line;

FIG. 23 is a diagram showing a northern blotting result obtained by hybridizing the same sample as in FIG. 22 with β-actin probe;

FIG. 24(a) is a diagram showing a northern blotting result to determine whether or not the MIG3 protooncogene of the present invention is expressed in a normal human 12-lane multiple tissues, and FIG. 24(b) is a diagram showing a northern blotting result obtained by hybridizing the same sample as in FIG. 24(a) with β-actin probe;

FIG. 25 is a diagram showing a northern blotting result to determine whether or not the MIG8 protooncogene of the present invention is expressed in a normal human 12-lane multiple tissues;

FIG. 26 is a diagram showing a northern blotting result obtained by hybridizing the same sample as in FIG. 25 with β-actin probe;

FIG. 27(a) is a diagram showing a northern blotting result to determine whether or not the MIG10 protooncogene of the present invention is expressed in a normal human 12-lane multiple tissues, and FIG. 27(b) is a diagram showing a northern blotting result obtained by hybridizing the same sample as in FIG. 27(a) with β-actin probe;

FIG. 28(a) is a diagram showing a northern blotting result to determine whether or not the MIG13 protooncogene of the present invention is expressed in a normal human 12-lane multiple tissues, and FIG. 28(b) is a diagram showing a northern blotting result obtained by hybridizing the same sample as in FIG. 28(a) with β-actin probe;

FIG. 29(a) is a diagram showing a northern blotting result to determine whether or not the MIG14 protooncogene of the present invention is expressed in a normal human 12-lane multiple tissues, and FIG. 29(b) is a diagram showing a northern blotting result obtained by hybridizing the same sample as in FIG. 29(a) with β-actin probe;

FIG. 30 is a diagram showing a northern blotting result to determine whether or not the MIG18 protooncogene of the present invention is expressed in a normal human 12-lane multiple tissues;

FIG. 31 is a diagram showing a northern blotting result obtained by hybridizing the same sample as in FIG. 30 with β-actin probe;

FIG. 32 is a diagram showing a northern blotting result to determine whether or not the MIG19 protooncogene of the present invention is expressed in a normal human 12-lane multiple tissues;

FIG. 33 is a diagram showing a northern blotting result obtained by hybridizing the same sample as in FIG. 32 with β-actin probe;

FIG. 34 is a diagram showing a northern blotting result to determine whether or not the MIG5 protooncogene of the present invention is expressed in a normal human 12-lane multiple tissues;

FIG. 35 is a diagram showing a northern blotting result obtained by hybridizing the same sample as in FIG. 34 with β-actin probe;

FIG. 36 is a diagram showing a northern blotting result to determine whether or not the MIG7 protooncogene of the present invention is expressed in a normal human 12-lane multiple tissues;

FIG. 37 is a diagram showing a northern blotting result obtained by hybridizing the same sample as in FIG. 36 with β-actin probe;

FIG. 38(a) is a diagram showing a northern blotting result to determine whether or not the MIG3 protooncogene of the present invention is expressed in the human cancer cell lines, and FIG. 38(b) is a diagram showing a northern blotting result obtained by hybridizing the same sample as in FIG. 38(a) with β-actin probe;

FIG. 39 is a diagram showing a northern blotting result to determine whether or not the MIG8 protooncogene of the present invention is expressed in the human cancer cell lines;

FIG. 40 is a diagram showing a northern blotting result obtained by hybridizing the same sample as in FIG. 39 with β-actin probe;

FIG. 41(a) is a diagram showing a northern blotting result to determine whether or not the MIG10 protooncogene of the present invention is expressed in the human cancer cell lines, and FIG. 41(b) is a diagram showing a northern blotting result obtained by hybridizing the same sample as in FIG. 41(a) with β-actin probe;

FIG. 42(a) is a diagram showing a northern blotting result to determine whether or not the MIG13 protooncogene of the present invention is expressed in the human cancer cell lines, and FIG. 42(b) is a diagram showing a northern blotting result obtained by hybridizing the same sample as in FIG. 42(a) with β-actin probe;

FIG. 43(a) is a diagram showing a northern blotting result to determine whether or not the MIG14 protooncogene of the present invention is expressed in the human cancer cell lines, and FIG. 43(b) is a diagram showing a northern blotting result obtained by hybridizing the same sample as in FIG. 43(a) with β-actin probe;

FIG. 44 is a diagram showing a northern blotting result to determine whether or not the MIG 18 protooncogene of the present invention is expressed in the human cancer cell lines;

FIG. 45 is a diagram showing a northern blotting result obtained by hybridizing the same sample as in FIG. 44 with β-actin probe;

FIG. 46 is a diagram showing a northern blotting result to determine whether or not the MIG19 protooncogene of the present invention is expressed in the human cancer cell lines;

FIG. 47 is a diagram showing a northern blotting result obtained by hybridizing the same sample as in FIG. 46 with β-actin probe;

FIG. 48 is a diagram showing a northern blotting result to determine whether or not the MIG5 protooncogene of the present invention is expressed in the human cancer cell lines;

FIG. 49 is a diagram showing a northern blotting result obtained by hybridizing the same sample as in FIG. 48 with β-actin probe;

FIG. 50 is a diagram showing a northern blotting result to determine whether or not the MIG7 protooncogene of the present invention is expressed in the human cancer cell lines;

FIG. 51 is a diagram showing a northern blotting result obtained by hybridizing the same sample as in FIG. 50 with β-actin probe; and

FIGS. 52 to 60 are diagrams showing results of sodium dodecyl sulfate-polyacrylamide gel electrophoresis (SDS-PAGE) to determine sizes of the proteins expressed before and after L-arabinose induction after the MIG3, MIG8, MIG10, MIG18, MIG13, MIG14, MIG19, MIG5 and MIG7 protooncogenes of the present invention are transformed into Escherichia coli, respectively.

### BEST MODES FOR CARRYING OUT THE INVENTION

Hereinafter, preferred embodiments of the present invention will be described in detail referring to the accompanying drawings.

1. MIG3

The protooncogene, human migration-inducing gene 3 (MIG3), of the present invention (hereinafter, referred to as MIG3 protooncogene) has a 2,295-bp full-length DNA sequence set forth in SEQ ID NO: 1.

In the DNA sequence of SEQ ID NO: 1, the open reading frame corresponding to nucleotide sequence positions from 89 to 709 (707-709: a stop codon) is a full-length protein coding region, and an amino acid sequence derived from the protein coding region is set forth in SEQ ID NO: 2 and contains 206 amino acids (hereinafter, referred to as “MIG3 protein”).

The DNA sequence of SEQ ID NO: 1 has been deposited with Accession No. AY239293 into the GenBank database of U.S. National Institutes of Health (NIH) (Publication Date: Dec. 31, 2004), and the DNA sequencing result revealed that its DNA sequence was similar to that of the Homo sapiens cDNA: FLJ23513 fis, clone LNG03869 gene deposited with Accession No. AK027166 into the database. A protein expressed from the protooncogene of the present invention contains 206 amino acids and has an amino acid sequence set forth in SEQ ID NO: 2 and a molecular weight of approximately 23 kDa.

2. MIG8

The protooncogene, human migration-inducing gene 8 (MIG8), of the present invention (hereinafter, referred to as MIG8 protooncogene) has a 3,737-bp full-length DNA sequence set forth in SEQ ID NO: 5.

In the DNA sequence of SEQ ID NO: 5, the open reading frame corresponding to nucleotide sequence positions from 113 to 1627 (1625-1627: a stop codon) is a full-length protein coding region, and an amino acid sequence derived from the protein coding region is set forth in SEQ ID NO: 6 and contains 665 amino acids (hereinafter, referred to as “MIG8 protein”).

The DNA sequence of SEQ ID NO: 5 has been deposited with Accession No. AY311389 into the GenBank database of U.S. National Institutes of Health (NIH) (Publication Date: Dec. 31, 2004), and the DNA sequencing result revealed that its amino acid sequence was identical with that of the Homo sapiens apoptosis inhibitor 5 (API5) gene deposited with Accession No. NM_{—}006595 and NM_{—}021112 into the database, but some of its DNA sequence was different to that of the Homo sapiens apoptosis inhibitor 5 (API5) gene.

A protein expressed from the protooncogene of the present invention contains 504 amino acids and has an amino acid sequence set forth in SEQ ID NO: 6 and a molecular weight of approximately 57 kDa.

3. MIG10

The protooncogene, human migration-inducing gene 10 (MIG10), of the present invention (hereinafter, referred to as MIG10 protooncogene) has a 1,321-bp full-length DNA sequence set forth in SEQ ID NO: 9.

In the DNA sequence of SEQ ID NO: 9, the open reading frame corresponding to nucleotide sequence positions from 23 to 1276 (1274-1276: a stop codon) is a full-length protein coding region, and an amino acid sequence derived from the protein coding region is set forth in SEQ ID NO: 10 and contains 417 amino acids (hereinafter, referred to as “MIG10 protein”).

The DNA sequence of SEQ ID NO: 9 has been deposited with Accession No. AY423725 into the GenBank database of U.S. National Institutes of Health (NIH) (Publication Date: Dec. 31, 2004), and the DNA sequencing result revealed that its DNA sequence was identical with those of the Homo sapiens phosphoglycerate kinase 1 gene and the Homo sapiens phosphoglycerate kinase 1 (PGK1) gene, deposited with Accession No. BC023234 and NM_{—}000291 into the database, respectively.

A protein expressed from the protooncogene of the present invention contains 417 amino acids and has an amino acid sequence set forth in SEQ ID NO: 10 and a molecular weight of approximately 45 kDa.

4. MIG3

The protooncogene, human migration-inducing gene 13 (MIG13), of the present invention (hereinafter, referred to as MIG13 protooncogene) has a 1,019-bp full-length DNA sequence set forth in SEQ ID NO: 13.

In the DNA sequence of SEQ ID NO: 13, the open reading frame corresponding to nucleotide sequence positions from 11 to 844 (842-844: a stop codon) is a full-length protein coding region, and an amino acid sequence derived from the protein coding region is set forth in SEQ ID NO: 14 and contains 277 amino acids (hereinafter, referred to as “MIG13 protein”).

The DNA sequence of SEQ ID NO: 13 has been deposited with Accession No. AY336090 into the GenBank database of U.S. National Institutes of Health (NIH) (Publication Date: Dec. 31, 2004), and the DNA sequencing result revealed that some of its DNA sequence was similar to that of the gene of full-length cDNA clone CS0DL001YE02 of B cells (Ramos cell line) Cot 25-normalized of Homo sapiens (human) deposited with Accession No. CR613087 into the database.

A protein expressed from the protooncogene of the present invention contains 277 amino acids and has an amino acid sequence set forth in SEQ ID NO: 14 and a molecular weight of approximately 31 kDa.

5. MIG14

The protooncogene, human migration-inducing gene 14 (MIG14), of the present invention (hereinafter, referred to as MIG14 protooncogene) has a 1,142-bp full-length DNA sequence set forth in SEQ ID NO: 17.

In the DNA sequence of SEQ ID NO: 17, the open reading frame corresponding to nucleotide sequence positions from 67 to 1125 (1123-1125: a stop codon) is a full-length protein coding region, and an amino acid sequence derived from the protein coding region is set forth in SEQ ID NO: 18 and contains 206 amino acids (hereinafter, referred to as “MIG14 protein”).

The DNA sequence of SEQ ID NO: 17 has been deposited with Accession No. AY336091 into the GenBank database of U.S. National Institutes of Health (NIH) (Publication Date: Dec. 31, 2004), and the DNA sequencing result revealed that its DNA sequence was identical with those of the genes of the Homo sapiens RAE1 RNA export 1 homolog (S. pombe) (RAE1) and the full-length cDNA clone CS0DI002YP18 of Placenta Cot 25-normalized of Homo sapiens (human), deposited with Accession No. NM_{—}003610 and CR626728 into the database, respectively.

A protein expressed from the protooncogene of the present invention contains 352 amino acids and has an amino acid sequence set forth in SEQ ID NO: 18 and a molecular weight of approximately 39 kDa.

6. MIG18

The protooncogene, human migration-inducing gene 18 (MIG18), of the present invention (hereinafter, referred to as MIG18 protooncogene) has a 3,633-bp full-length DNA sequence set forth in SEQ ID NO: 21.

In the DNA sequence of SEQ ID NO: 21, the open reading frame corresponding to nucleotide sequence positions from 215 to 2212 (2210-2212: a stop codon) is a full-length protein coding region, and an amino acid sequence derived from the protein coding region is set forth in SEQ ID NO: 22 and contains 665 amino acids (hereinafter, referred to as “MIG18 protein”).

The DNA sequencing result revealed that the MIG18 protooncogene of the present invention had the same protein sequence as the Homo sapiens SH3-domain kinase binding protein 1 (SH3KBP1) (GenBank Accession No. NM_{—}031892) (Take, H., et al., Biochem. Biophy. Res. Comm. 268: 321-328, 2000) that functions to transduce signals associated with the epidermal growth factor by binding to the c-Cb1 gene (Langdon, W. Y., et al., Proc. Natl. Acad. Sci USA 86: 1168-1172, 1989), but some of its DNA sequence was different to that of the gene the Homo sapiens SH3-domain kinase binding protein 1.

A protein expressed from the protooncogene of the present invention contains 665 amino acids and has an amino acid sequence set forth in SEQ ID NO: 22 and a molecular weight of approximately 73 kDa.

7. MIG19

The protooncogene, human migration-inducing gene 19 (MIG19), of the present invention (hereinafter, referred to as MIG19 protooncogene) has a 4,639-bp full-length DNA sequence set forth in SEQ ID NO: 25.

In the DNA sequence of SEQ ID NO: 25, the open reading frame corresponding to nucleotide sequence positions from 65 to 2965 (2963-2965: a stop codon) is a full-length protein coding region, and an amino acid sequence derived from the protein coding region is set forth in SEQ ID NO: 26 and contains 966) amino acids (hereinafter, referred to as “MIG19 protein”).

The DNA sequence of SEQ ID NO: 25 has been deposited with Accession No. AY450308 into the GenBank database of U.S. National Institutes of Health (NIH) (Publication Date: Dec. 31, 2004), and the DNA sequencing result revealed that some of its protein sequence was identical with that of the Homo sapiens membrane component, chromosome 17, surface marker 2 (ovarian carcinoma antigen CA125) (M17S2), transcript variant 3 gene deposited with Accession No. NM_{—}031862 into the database, but some of its DNA sequence was different to that of the said gene.

A protein expressed from the protooncogene of the present invention contains 966 amino acids and has an amino acid sequence set forth in SEQ ID NO: 26 and a molecular weight of approximately 107 kDa.

8. MIG5

The protooncogene, human migration-inducing gene 5 (MIG5), of the present invention (hereinafter, referred to as MIG5 protooncogene) has a 833-bp full-length DNA sequence set forth in SEQ ID NO: 29.

In the DNA sequence of SEQ ID NO: 29, the open reading frame corresponding to nucleotide sequence positions from 159 to 737 (735-737: a stop codon) is a full-length protein coding region, and an amino acid sequence derived from the protein coding region is set forth in SEQ ID NO: 30 and contains 192 amino acids (hereinafter, referred to as “MIG5 protein”).

The DNA sequence of SEQ ID NO: 29 has been deposited with Accession No. AY279384 into the GenBank database of U.S. National Institutes of Health (NIH) (Publication Date: Dec. 31, 2004), and the DNA sequencing result revealed that its DNA sequence was identical with that of the Homo sapiens ras-related C3 botulinum toxin substrate 1 (rho family, small GTP binding protein Rac1) (RAC1), transcript variant Rac1 gene deposited with Accession No. NM_{—}006908 into the database, respectively.

A protein expressed from the protooncogene of the present invention contains 192 amino acids and has an amino acid sequence set forth in SEQ ID NO: 30 and a molecular weight of approximately 21 kDa.

9. MIG7

The protooncogene, human migration-inducing gene 7 (MIG7), of the present invention (hereinafter, referred to as MIG7 protooncogene) has a 2,364-bp full-length DNA sequence set forth in SEQ ID NO: 33.

In the DNA sequence of SEQ ID NO: 33, the open reading frame corresponding to nucleotide sequence positions from 1435 to 1685 (1683-1685: a stop codon) is a full-length protein coding region, and an amino acid sequence derived from the protein coding region is set forth in SEQ ID NO: 34 and contains 76 amino acids (hereinafter, referred to as “MIG7 protein”).

The DNA sequence of SEQ ID NO: 33 has been deposited with Accession No. AY305872 into the GenBank database of U.S. National Institutes of Health (NIH) (Publication Date: Dec. 31, 2004), and the DNA sequencing result revealed that some of its DNA sequence was identical with those of the genes of the Homo sapiens T cell receptor alpha delta locus (TCRA/TCRD) on chromosome 14 deposited with Accession No. NG_{—}001332, the Homo sapiens T-cell receptor alpha delta locus from bases 1 to 250529 (section 1 of 5) of the Complete Nucleotide Sequence deposited with Accession No. AE000658, AE000521 and U85195, and the Homo sapiens (N6-adenosine)-methyltransferase gene deposited with Accession No. AF283991 into the database, respectively.

A protein expressed from the protooncogene of the present invention contains 76 amino acids and has an amino acid sequence set forth in SEQ ID NO: 34 and a molecular weight of approximately 9 kDa.

Meanwhile, because of degeneracy of codons, or considering preference of codons for living organisms to express the protooncogenes, the protooncogenes of the present invention may be variously modified in coding regions without changing an amino acid sequence of the oncogenic protein expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the protooncogene; and fragments of the protooncogene. The term “substantially the same polynucleotide” means DNA encoding the same translated protein product and having DNA sequence homology of at least 80%, preferably at least 90%, and the most preferably at least 95% with the protooncogene of the present invention.

Also, one or more amino acids may be substituted, added or deleted in the amino acid sequence of the protein within a range that does not affect functions of the protein, and only some portion of the protein may be used depending on its usage. Such a modified amino acid sequence is also included in the scope of the present invention. Accordingly, the present invention also includes a polypeptide having substantially the same amino acid sequence as the oncogenic protein; and fragments of the protein. The term “substantially the same polypeptide” means a polypeptide having sequence homology of at least 80%, preferably at least 90%, and the most preferably at least 95%.

The protooncogenes and proteins of the present invention may be separated from human cancer tissues, or be synthesized according to the known methods for synthesizing DNA or peptide. Also, the gene prepared thus may be inserted into a vector for expression in microorganisms, already known in the art, to obtain an expression vector, and then the expression vector may be introduced into suitable host cells, for example Escherichia coli, yeast cells, etc. DNA of the gene of the present invention may be replicated in a large quantity or its protein may be produced in a commercial quantity in such a transformed host.

Upon constructing the expression vector, expression regulatory sequences such as a promoter and a terminator, autonomously replicating sequences, secretion signals, etc. may be suitably selected and combined depending on kinds of the host cells that produce the gene or the protein.

The genes of the present invention are proved to be strong oncogenes capable of developing the lung cancer since it was revealed the gene was hardly expressed in a normal lung tissue, but overexpressed in a lung cancer tissue and a lung cancer cell line in the analysis methods such as a northern blotting, etc. Also, the genes are proved to be a cancer metastasis-related gene capable of inducing cancer metastasis, considering that its expression is increased in the metastatic lymph node cancer tissues. In addition to the epithelial tissue such as the lung cancer, the protooncogenes of the present invention are highly expressed in other cancerous tumor tissues such as leukemia, uterine cancer, lymphoma, colon cancer, skin cancer, etc. Accordingly, the protooncogenes of the present invention are considered to be common oncogenes in the various oncogenesis, and may be effectively used for diagnosing the various cancers and producing the transformed animals.

For example, a method for diagnosing the cancer using the protooncogenes includes a step of determining whether or not a subject has the protooncogenes of the present invention by detecting the protooncogenes in the various methods known in the art after all or some of the protooncogenes are used as proves and hybridized with nucleic acid extracted from the subject's body fluids. It can be easily confirmed that the genes are present in the tissue samples by using the probes labeled with a radioactive isotope, an enzyme, etc. Accordingly, the present invention provides kits for diagnosing the cancer containing all or some of the protooncogenes.

The transformed animals may be obtained by introducing the protooncogenes of the present invention into mammals, for example rodents such as a rat, and the protooncogenes are preferably introduced at the fertilized egg stage prior to at least 8-cell stage. The transformed animals prepared thus may be effectively used for searching carcinogenic substances or anticancer substances such as antioxidants.

The proteins derived from the protooncogenes of the present invention may be effectively used for producing antibodies as a diagnostic tool. The antibodies of the present invention may be produced as the monoclonal or polyclonal antibodies according to the conventional methods known in the art using the proteins expressed from the protooncogenes of the present invention; or their fragments, and therefore such a antibody may be used to diagnose the cancer and the cancer metastasis by determining whether or not the proteins are expressed in the body fluid samples of the subject using the method known in the art, for example an enzyme linked immunosorbent assay (ELISA), a radioimmunoassay (RIA), a sandwich assay, western blotting or immunoblotting on the polyacrylamide gel, etc.

Also, the protooncogene of the present invention may be used to establish cancer cell lines that can continue to grow in an uncontrolled manner, and such a cell line may be, for example, produced from the tumorous tissue developed in the back of a nude mouse using fibroblast cell transfected with the protooncogenes. Such a cancer cell line may be effectively used for searching anticancer agents, etc.

Hereinafter, the present invention will be described in detail referring to preferred examples.

However, the description proposed herein is just a preferable example for the purpose of illustrations only, not intended to limit the scope of the invention.

### EXAMPLE 1

### Cultivation of Tumor Cell and Separation of Total RNA

1-1: MIG3, MIG10, MIG13 and MIG14

(Step 1) Cultivation of Tumor Cell

In order to conduct the mRNA differential display method, a normal lung tissue was obtained, and a primary lung cancer tissue and a cancer tissue metastasized to the right lung were obtained from a lung cancer patient who has not been previously subject to the anticancer and/or radiation therapies upon surgery operation. A549 (American Type Culture Collection; ATCC Number CCL-185) was used as the human lung cancer cell line in the differential display method.

Cells obtained from the obtained tissues and the A549 lung cancer cell line were grown in a Waymouth's MB 752/1 medium (Gibco) containing 2 mM glutamine, 100 IU/ml penicillin, 100 μg/ml streptomycin and 10% fetal bovine serum (Gibco, U.S.). The culture cells used in this experiment are cells at the exponentially growing stage, and the cells showing a viability of at least 95% by a trypan blue dye exclusion test were used herein (Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal lung tissue, the primary lung cancer tissue, the metastatic lung cancer tissue and the A549 cell, each obtained in Step 1, using the commercially available system RNeasy total RNA kit (Qiagen Inc., Germany), and then DNA contaminants were removed from the RNA samples using the message clean kit (GenHunter Corp., Brookline, Mass., U.S.).

1-2: MIG8, MIG18, MIG19, MIG5 and MIG9

(Step 1) Cultivation of Tumor Cell

In order to conduct the mRNA differential display method, a normal exocervical tissue was obtained from a patient suffering from an uterine myoma who has been subject to hysterectomy, and a primary cervical tumor tissue and a metastatic lymph node tumor tissue were obtained from an uterine cancer patient the who has not been previously subject to the anticancer and/or radiation therapies upon surgery operation. CUMC-6 (Kim, J. W. et al., Gynecol. Oncol. 62: 230-240, 1996) was used as the human cervical cancer cell line in the differential display method.

Cells obtained from the obtained tissues and the CUMC-6 cell line were grown in a Waymouth's MB 752/1 medium (Gibco) containing 2 mM glutamine, 100 IU/ml penicillin, 100 μg/ml streptomycin and 10% fetal bovine serum (Gibco, U.S.). The culture cells used in this experiment are cells at the exponentially growing stage, and the cells showing a viability of at least 95% by a trypan blue dye exclusion test were used herein (Freshney, “Culture of Animal Cells: A Manual of Basic Technique” 2nd Ed., A. R. Liss, New York, 1987).

(Step 2) Separation of RNA and mRNA Differential Display Method

The total RNA samples were separated from the normal exocervical tissue, the primary cervical tumor tissue, the metastatic lymph node tumor tissue and the CUMC-6 cell, each obtained in Step 1, using the commercially available system RNeasy total RNA kit (Qiagen Inc., Germany), and then DNA contaminants were removed from the RNA samples using the message clean kit (GenHunter Corp., Brookline, Mass., U.S.).

### EXAMPLE 2

### Differential Display Reverse Transcription-Polymerase Chain Reaction (DDRT-PCR)

2-1: MIG3

The differential display reverse transcription was carried out using a slightly modified reverse transcription-polymerase chain reaction (RT-PCR) proposed by Liang, P. and A. B. Pardee.

At first, reverse transcription was conducted on 0.2 μg of each of the total RNAs obtained in Step 1 of Example 1-1 using an anchored primer H-T11A (5-AAGCTTTTTTTTTTTC-3′, RNAimage kit, Genhunter, Cor., MA, U.S.) having a DNA sequence set forth in SEQ ID NO: 3 as the anchored oligo-dT primer.

Then, a PCR reaction was carried out in the presence of 0.5 mM [α-³⁵S] dATP (1200 Ci/mmole) using the same anchored primer and the primer H-AP22 (5′-AAGCTTTTGATCC-3′) having a DNA sequence set forth in SEQ ID NO: 4 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 305-base pair (bp) band with L276-811 cDNA (Base positions from 1862 to 2166 of SEQ ID NO: 1) was cut out from the dried gel. The extracted gel was heated for 15 minutes to elute the L276-811 cDNA, and then the PCR reaction was repeated with the same primer under the same condition as described above to re-amplify the L276-811 cDNA, except that [α-³⁵S]-labeled dATP (1200 Ci/mmole) and 20 μM dNTP were not used herein.

2-2: MIG8

The differential display reverse transcription was carried out using a slightly modified reverse transcription-polymerase chain reaction (RT-PCR) proposed by Liang, P. and A. B. Pardee.

At first, reverse transcription was conducted on 0.2 μg of each of the total RNAs obtained in Step 1 of Example 1-2 using an anchored primer H-T11C (5-AAGCTTTTTTTTTTTC-3′, RNAimage kit, Genhunter, Cor., MA, U.S.) having a DNA sequence set forth in SEQ ID NO: 7 as the anchored oligo-dT primer.

Then, a PCR reaction was carried out in the presence of 0.5 mM [α-³⁵S] dATP (1200 Ci/mmole) using the same anchored primer and the primer H-AP23 (5′-AAGCTTGGCTATG-3′) having a DNA sequence set forth in SEQ ID NO: 8 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 342-base pair (bp) band with CC231 cDNA (Base positions from 3142 to 3483 of SEQ ID NO: 5) was cut out from the dried gel. The extracted gel was heated for 15 minutes to elute the CC231 cDNA, and then the PCR reaction was repeated with the same primer under the same condition as described above to re-amplify the CC231 cDNA, except that [α-³⁵S]-labeled dATP (1200 Ci/mmole) and 20 μM dNTP were not used herein.

2-3: MIG10

At first, reverse transcription was conducted on 0.2 μg of each of the total RNAs obtained in Step 1 of Example 1-1 using an anchored primer H-T11C (5-AAGCTTTTTTTTTTTC-3′, RNAimage kit, Genhunter, Cor., MA, U.S.) having a DNA sequence set forth in SEQ ID NO: 11 as the anchored oligo-dT primer.

Then, a PCR reaction was carried out in the presence of 0.5 mM [α-³⁵S] dATP (1200 Ci/mmole) using the same anchored primer and the primer H-AP23 (5′-AAGCTTGGCTATG-3′) having a DNA sequence set forth in SEQ ID NO: 12 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 284-base pair (bp) band with L789 cDNA (Base positions from 1022 to 1305 of SEQ ID NO: 9) was cut out from the dried gel. The extracted gel was heated for 15 minutes to elute the L789 cDNA, and then the PCR reaction was repeated with the same primer under the same condition as described above to re-amplify the L789 cDNA, except that [α-³⁵S]-labeled dATP (1200 Ci/mmole) and 20 μM dNTP were not used herein.

2-4: MIG13

At first, reverse transcription was conducted on 0.2 μg of each of the total RNAs obtained in Step 1 of Example 1 using an anchored primer H-T11C (5-AAGCTTTTTTTTTTTC-3′, RNAimage kit, Genhunter, Cor., MA, U.S.) having a DNA sequence set forth in SEQ ID NO: 15 as the anchored oligo-dT primer.

Then, a PCR reaction was carried out in the presence of 0.5 mM [α-³⁵S] dATP (1200 Ci/mmole) using the same anchored primer and the primer H-AP21 (5′-AAGCTTTCTCTGG-3′) having a DNA sequence set forth in SEQ ID NO: 16 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40 ° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 295-base pair (bp) band with L986 cDNA (Base positions from 685 to 979 of SEQ ID NO: 13) was cut out from the dried gel. The extracted gel was heated for 15 minutes to elute the L986 cDNA, and then the PCR reaction was repeated with the same primer under the same condition as described above to re-amplify the L986 cDNA, except that [α-³⁵S]-labeled dATP (1200 Ci/mmole) and 20 μM dNTP were not used herein.

2-5: MIG14

At first, reverse transcription was conducted on 0.2 μg of each of the total RNAs obtained in Step 1 of Example 1 using an anchored primer H-T11A (5-AAGCTTTTTTTTTTTA-3′, RNAimage kit, Genhunter, Cor., MA, U.S.) having a DNA sequence set forth in SEQ ID NO: 19 as the anchored oligo-dT primer.

Then, a PCR reaction was carried out in the presence of 0.5 mM [α-³⁵S] dATP (1200 Ci/mmole) using the same anchored primer and the primer H-AP21 (5′-AAGCTTTCTCTGG-3′) having a DNA sequence set forth in SEQ ID NO: 20 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 276-base pair (bp) band with L1284 cDNA (Base positions from 823 to 1098 of SEQ ID NO: 17) was cut out from the dried gel. The extracted gel was heated for 15 minutes to elute the L1284 cDNAA, and then the PCR reaction was repeated with the same primer under the same condition as described above to re-amplify the L1284 cDNA, except that [α-³⁵S]-labeled dATP (1200 Ci/mmole) and 20 μM dNTP were not used herein.

2-6: MIG18

At first, reverse transcription was conducted on 0.2 μg of each of the total RNAs obtained in Step 1 of Example 1 using an anchored primer H-11A (5-AAGCTTTTTTTTTTTA-3′, RNAimage kit, Genhunter, Cor., MA, U.S.) having a DNA sequence set forth in SEQ ID NO: 23 as the anchored oligo-dT primer.

Then, a PCR reaction was carried out in the presence of 0.5 mM [α-³⁵S] dATP (1200 Ci/mmole) using the same anchored primer and the primer H-AP36 (5′-AAGCTTCGACGCT-3′) having a DNA sequence set forth in SEQ ID NO: 24 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 221-base pair (bp) band with CA367 cDNA (Base positions from 2920 to 3140 of SEQ ID NO: 21) was cut out from the dried gel. The extracted gel was heated for 15 minutes to elute the CA367 cDNA, and then the PCR reaction was repeated with the same primer under the same condition as described above to re-amplify the CA367 cDNA, except that [α-³⁵S]-labeled dATP (1200 Ci/mmole) and 20 μM dNTP were not used herein.

2-7: MIG19

At first, reverse transcription was conducted on 0.2 μg of each of the total RNAs obtained in Step 1 of Example 1 using an anchored primer H-T11A (5-AAGCTTTTTTTTTTTA-3′, RNAimage kit, Genhunter, Cor., MA, U.S.) having a DNA sequence set forth in SEQ ID NO: 27 as the anchored oligo-dT primer.

Then, a PCR reaction was carried out in the presence of 0.5 mM [α-³⁵S] dATP (1200 Ci/mmole) using the same anchored primer and the primer H-AP33 (5′-AAGCTTGCTGCTC-3′) having a DNA sequence set forth in SEQ ID NO: 28 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 381-base pair (bp) band with CA335 cDNA (Base positions from 4123 to 4503 of SEQ ID NO: 25) was cut out from the dried gel. The extracted gel was heated for 15 minutes to elute the CA335 cDNA, and then the PCR reaction was repeated with the same primer under the same condition as described above to re-amplify the CA335 cDNA, except that [α-³⁵S]-labeled dATP (1200 Ci/mmole) and 20 μM dNTP were not used herein.

2-8: MIG5

The differential display reverse transcription was carried out using a slightly modified reverse transcription-polymerase chain reaction (RT-PCR) proposed by Liang, P. and A. B. Pardee.

At first, reverse transcription was conducted on 0.2 μg of each of the total RNAs obtained in Step 1 of Example 1 using an anchored primer H-T11G (5-AAGCTTTTTTTTTTTG-3′, RNAimage kit, Genhunter, Cor., MA, U.S.) having a DNA sequence set forth in SEQ ID NO: 31 as the anchored oligo-dT primer.

Then, a PCR reaction was carried out in the presence of 0.5 mM [α-³⁵S] dATP (1200 Ci/mmole) using the same anchored primer and the primer H-AP26 (5′-AAGCTTGCCATGG-3′) having a DNA sequence set forth in SEQ ID NO: 32 among the random 5′-11-mer primers (RNAimage primer sets 1-5) H-AP 1 to 40. The PCR reaction was conducted under the following conditions: the total 40 amplification cycles consisting of a denaturation step at 95 ° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 263-base pair (bp) band with CG263 cDNA (Base positions from 476 to 738 of SEQ ID NO: 29) was cut out from the dried gel. The extracted gel was heated for 15 minutes to elute the CG263 cDNA, and then the PCR reaction was repeated with the same primer under the same condition as described above to re-amplify the CG263 cDNA, except that [α-³⁵S]-labeled dATP (1200 Ci/mmole) and 20 μM dNTP were not used herein.

2-9: MIG7

The differential display reverse transcription was carried out using a modified reverse transcription-polymerase chain reaction (RT-PCR) proposed by Liang, P. and A. B. Pardee.

At first, reverse transcription was conducted on 0.2 μg of each of the total RNAs obtained in Step 1 of Example 1 using an anchored primer H-T11G (5-AAGCTTTTTTTTTTTG-3′, RNAimage kit, Genhunter, Cor., MA, U.S.) having a DNA sequence set forth in SEQ ID NO: 35 as the anchored oligo-dT primer.

Then, a PCR reaction was carried out in the presence of 0.5 mM [α-³⁵S] dATP (1200 Ci/mmole) using the same anchored primer and the primer H-AP23 (5′-AAGCTTGGCTATG-3′) having a DNA sequence set forth in SEQ ID NO: 36 among the random 5′-11-mer primers (RNAimage primer sets 1-5) H-AP 1 to 40. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one final extension step at 72° C. for 5 minutes.

The fragments amplified in the PCR reaction were dissolved in a 6% polyacrylamide sequencing gel for DNA sequence, and then a position of a differentially expressed band was confirmed using autoradiography.

A 327-base pair (bp) band with CG233 cDNA (Base positions from 1903 to 2229 of SEQ ID NO: 33) was cut out from the dried gel. The extracted gel was heated for 15 minutes to elute the CG233 cDNA, and then the PCR reaction was repeated with the same primer under the same condition as described above to re-amplify the CG233 cDNA, except that [α-³⁵S]-labeled dATP (1200 Ci/mmole) and 20 μM dNTP were not used herein.

### EXAMPLE 3

### Cloning

The L276-811 PCR product; the CC231 PCR product; the L789 PCR product; the L986 PCR product; the L1284 PCR product; the CA367 PCR product; the CA335 PCR product; the CG263 PCR product; and the CG233 PCR product, which were all re-amplified as described above, were inserted into a pGEM-T EASY vector, respectively, according to the manufacturer's manual using the TA cloning system (Promega, U.S.).

(Step 1) Ligation Reaction

2 μl of each of the L276-811 PCR product; the CC231 PCR product; the L789 PCR product; the L986 PCR product; the L1284 PCR product; the CA367 PCR product; the CA335 PCR product; the CG263 PCR product and the CG233 PCR product, which were all re-amplified in Example 2, 1 μl of pGEM-T EASY vector (50 ng), 1 μl of T4 DNA ligase (10× buffer) and 1 μl of T4 DNA ligase (3 weiss units/μl; Promega) were put into a 0.5 ml test tube, and distilled water was added thereto to a final volume of 10 μl. The ligation reaction mixtures were incubated overnight at 14° C.

(Step 2) Transformation of TA Clone

E. coli JM109 (Promega, WI, U.S.) was incubated in 10 ml of LB broth (10 g of bacto-tryptone, 5 g of bacto-yeast extract, 5 g of NaCl) until the optical density at 600 nm reached approximately 0.3 to 0.6. The incubated mixture was kept in ice at about 10 minutes and centrifuged at 4,000 rpm for 10 minutes at 4° C., and then the supernatant wad discarded and the cell was collected. The collected cell pellet was exposed to 10 ml of 0.1 M ice-cold CaCl₂ for approximately 30 minutes to 1 hours to produce a competent cell. The product was centrifuged again at 4,000 rpm for 10 minutes at 4° C., and then the supernatant wad discarded and the cell was collected and suspended in 2 ml of 0.1 M ice-cold CaCl₂.

200 μl of the competent cell suspension was transferred to a new microfuge, and 2 μl of the ligation reaction product prepared in Step 1 was added thereto. The resultant mixture was incubated in a water bath at 42° C. for 90 seconds, and then quenched at 0° C. 800 μl of SOC medium (2.0 g of bacto-tryptone, 0.5 g of bacto-yeast extract, 1 ml of 1 M NaCl, 0.25 ml of 1 M KCl, 97 ml of TDW, 1 ml of 2 M Mg²⁺, 1 ml of 2 M glucose) was added thereto and the resultant mixture was incubated at 37° C. for 45 minutes in a rotary shaking incubator at 220 rpm.

25 μl of X-gal (stored in 40 mg/ml of dimethylformamide) was spread with a glass rod on a LB plate supplemented with ampicillin and previously put into the incubator at 37° C., and 25 μl of transformed cell was added thereto and spread again with a glass rod, and then incubated overnight at 37° C. After incubation, the 3 to 4 formed white colonies was selected to seed-culture each of the selected cells in a LB plate supplemented with ampicillin. In order to construct a plasmid, the colonies considered to be colonies into which the ligation reaction products were introduced respectively, namely the transformed E. coli strains JM109/L276-811; JM109/CC231; JM109/L789; JM109/L986; JM109/L1284; JM109/CA367; JM109/CA335; JM109/CG263 and JM109/CG233 were selected and incubated in 10 ml of terrific broth (900 ml of TDW, 12 g of bacto-tryptone, 24 g of bacto-yeast extract, 4 ml of glycerol, 0.17 M KH₂PO₄, 100 ml of 0.72 N K₂HPO₄).

### EXAMPLE 4

### Separation of Recombinant Plasmid DNA

Each of the L276-811 plasmid DNA; the CC231 plasmid DNA; the L789 plasmid DNA; the L986 plasmid DNA; the L1284 plasmid DNA; the CA367 plasmid DNA; the CA335 plasmid DNA; the CG263 plasmid DNA and the CG233 plasmid DNA was separated from the transformed E. coli strains according to the manufacturer's manual using a Wizard™ Plus Minipreps DNA purification kit (Promega, U.S.).

It was confirmed that some of each of the separated plasmid DNAs was treated with a restriction enzyme ECoRI, and partial sequences of L276-811; CC231; L789; L986; L1284; CA367; CA335; CG263 and CG233 was inserted into the plasmid, respectively, by conducting electrophoresis in a 2% gel.

### EXAMPLE 5

### DNA Sequencing Analysis

5-1: MIG3

The L276-811 PCR product obtained in Example 2 was amplified, cloned, and then re-amplified according to the conventional method. The resultant L276-811 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1862 to 2166 of SEQ ID NO: 1, which is named “L276-811” in the present invention.

The 305-bp cDNA fragment obtained above, for example L276-811 was subject to the differential display reverse transcription-polymerase chain reaction (DDRT-PCR) using a 5′-random primer H-AP22 and a 3′-anchored primer H-T11A, and then confirmed using the electrophoresis.

As shown in FIG. 1, it was revealed from the differential display (DD) that the gene was differentially expressed in the normal lung tissue, the left lung cancer tissue, the metastatic lung cancer tissue metastasized from the left lung to the right lung, and the A549 lung cancer cell. As seen in FIG. 1, the 305-bp cDNA fragment L276-811 was expressed in the lung cancer tissue, the metastatic lung cancer tissue and the A549 lung cancer cell, but not expressed in the normal lung tissue. The L276-811 gene was the most highly expressed in the cancer tissue, particularly the metastatic cancer tissue.

5-2: MIG8

The CC231 PCR product obtained in Example 2 was amplified, cloned, and then re-amplified according to the conventional method. The resultant CC231 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 3142 to 3483 of SEQ ID NO: 5, which is named “CC231” in the present invention.

The 342-bp cDNA fragment obtained above, for example CC231 was subject to the differential display reverse transcription-polymerase chain reaction (DDRT-PCR) using a 5′-random primer H-AP23 and a 3′-anchored primer H-T11C, and then confirmed using the electrophoresis.

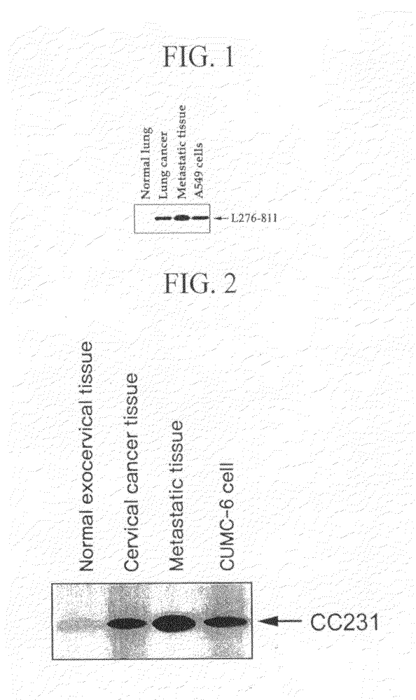

As shown in FIG. 2, it was revealed from the differential display (DD) that the gene was differentially expressed in the normal exocervical tissue, the metastatic lymph node tissue and the CUMC-6 cell. As seen in FIG. 2, the 342-bp cDNA fragment CC231 was expressed in the cervical cancer, the metastatic lymph node tissue and the CUMC-6 cancer cell, but not expressed in the normal tissue.

5-3: MIG10

The L789 PCR product obtained in Example 2 was amplified, cloned, and then re-amplified according to the conventional method. The resultant L789 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1022 to 1305 of SEQ ID NO: 9, which is named “L789” in the present invention.

The 284-bp cDNA fragment obtained above, for example L789 was subject to the differential display reverse transcription-polymerase chain reaction (DDRT-PCR) using a 5′-random primer H-AP23 and a 3′-anchored primer H-T11C, and then confirmed using the electrophoresis.

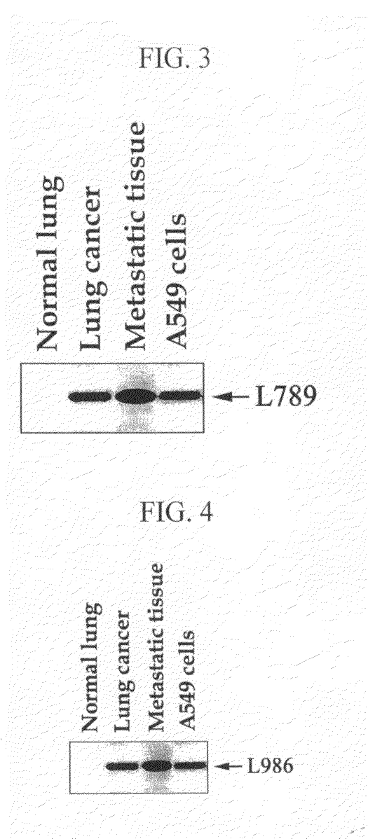

As shown in FIG. 3, it was revealed from the differential display (DD) that the gene was differentially expressed in the normal lung tissue, the left lung cancer tissue, the metastatic lung cancer tissue metastasized from the left lung to the right lung, and the A549 lung cancer cell. As seen in FIG. 3, the 255-bp cDNA fragment L276 was expressed in the lung cancer tissue, the metastatic lung cancer tissue and the A549 lung cancer cell, but not expressed in the normal lung tissue. The L276 gene was the most highly expressed in the cancer tissue, particularly the metastatic cancer tissue.

5-4: MIG13

The L986 PCR product obtained in Example 2 was amplified, cloned, and then re-amplified according to the conventional method. The resultant L986 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 685 to 979 of SEQ ID NO: 13, which is named “L986” in the present invention.

The 295-bp cDNA fragment obtained above, for example L986 was subject to the differential display reverse transcription-polymerase chain reaction (DDRT-PCR) using a 5′-random primer H-AP21 and a 3′-anchored primer H-T11C, and then confirmed using the electrophoresis.

As shown in FIG. 4, it was revealed from the differential display (DD) that the gene was differentially expressed in the normal lung tissue, the left lung cancer tissue, the metastatic lung cancer tissue metastasized from the left lung to the right lung, and the A549 lung cancer cell. As seen in FIG. 4, the 295-bp cDNA fragment L986 was expressed in the lung cancer tissue, the metastatic lung cancer tissue and the A549 lung cancer cell, but not expressed in the normal lung tissue. The L276-811 gene was the most highly expressed in the cancer tissue, particularly the metastatic cancer tissue.

5-5: MIG14

The L1284 PCR product obtained in Example 2 was amplified, cloned, and then re-amplified according to the conventional method. The resultant L1284 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 823 to 1098 of SEQ ID NO: 17, which is named “L1284” in the present invention.

The 276-bp cDNA fragment obtained above, for example L1284 was subject to the differential display reverse transcription-polymerase chain reaction (DDRT-PCR) using a 5′-random primer H-AP21 and a 3′-anchored primer H-T11A, and then confirmed using the electrophoresis.

As shown in FIG. 5, it was revealed from the differential display (DD) that the gene was differentially expressed in the normal lung tissue, the left lung cancer tissue, the metastatic lung cancer tissue metastasized from the left lung to the right lung, and the A549 lung cancer cell. As seen in FIG. 5, the 276-bp cDNA fragment L1284 was expressed in the lung cancer tissue, the metastatic lung cancer tissue and the A549 lung cancer cell, but not expressed in the normal lung tissue. The L1284 gene was the most highly expressed in the cancer tissue, particularly the metastatic cancer tissue.

5-6: MIG18

The CA367 PCR product obtained in Example 2 was amplified, cloned, and then re-amplified according to the conventional method. The resultant CA367 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 2920 to 3140 of SEQ ID NO: 21, which is named “CA367” in the present invention.

The 221-bp cDNA fragment obtained above, for example CA367 was subject to the differential display reverse transcription-polymerase chain reaction (DDRT-PCR) using a 5′-random primer H-AP36 and a 3′-anchored primer H-T11A, and then confirmed using the electrophoresis.

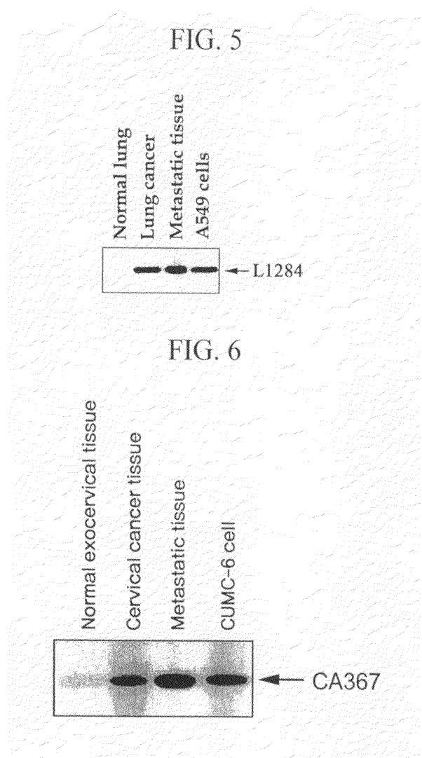

As shown in FIG. 6, it was revealed from the differential display (DD) that the gene was differentially expressed in the normal exocervical tissue, the metastatic lymph node tissue and the CUMC-6 cell. As seen in FIG. 6, the 221-bp cDNA fragment CA367 was expressed in the cervical cancer tissue, the metastatic lymph node tissue and the CUMC-6 cancer cell, but not expressed in the normal tissue.

5-7: MIG19

The CA335 PCR product obtained in Example 2 was amplified, cloned, and then re-amplified according to the conventional method. The resultant CA335 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 4123 to 4503 of SEQ ID NO: 25, which is named “CA335” in the present invention.

The 381-bp cDNA fragment obtained above, for example CA335 was subject to the differential display reverse transcription-polymerase chain reaction (DDRT-PCR) using a 5′-random primer H-AP33 and a 3′-anchored primer H-T11A, and then confirmed using the electrophoresis.

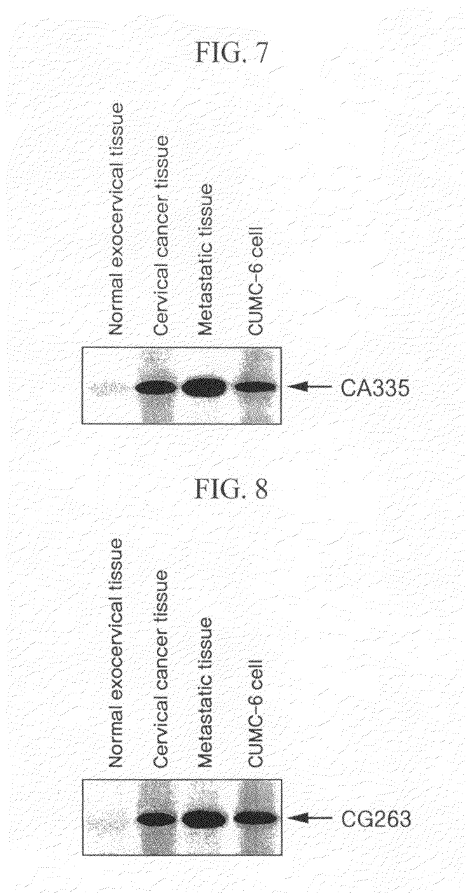

As shown in FIG. 7, it was revealed from the differential display (DD) that the gene was differentially expressed in the normal exocervical tissue, the metastatic lymph node tissue and the CUMC-6 cell. As seen in FIG. 7, the 381-bp cDNA fragment CA335 was expressed in the cervical cancer tissue, the metastatic lymph node tissue and the CUMC-6 cancer cell, but not expressed in the normal tissue.

5-8: MIG5

The CG263 PCR product obtained in Example 2 was amplified, cloned, and then re-amplified according to the conventional method. The resultant CG263 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 476 to 738 of SEQ ID NO: 29, which is named “CG263” in the present invention.

The 263-bp cDNA fragment obtained above, for example CG263 was subject to the differential display reverse transcription-polymerase chain reaction (DDRT-PCR) using a 5′-random primer H-AP26 and a 3′-anchored primer H-T11G, and then confirmed using the electrophoresis.

As shown in FIG. 8, it was revealed from the differential display (DD) that the gene was differentially expressed in the normal exocervical tissue, the metastatic lymph node tissue and the CUMC-6 cell. As seen in FIG. 8, the 263-bp cDNA fragment CG263 was expressed in the cervical cancer tissue, the metastatic lymph node tissue and the CUMC-6 cancer cell, but not expressed in the normal tissue.

5-9: MIG7

The CG233 PCR product obtained in Example 2 was amplified, cloned, and then re-amplified according to the conventional method. The resultant CG233 PCR fragment was sequenced according to a dideoxy chain termination method using the Sequenase version 2.0 DNA sequencing kit (United States Biochemical, Cleveland, Ohio, U.S.).

The DNA sequence of the said gene corresponds to nucleotide sequence positions from 1903 to 2229 of SEQ ID NO: 33, which is named “CG233” in the present invention.

The 327-bp cDNA fragment obtained above, for example CG233 was subject to the differential display reverse transcription-polymerase chain reaction (DDRT-PCR) using a 5′-random primer H-AP23 and a 3′-anchored primer H-T11G, and then confirmed using the electrophoresis.

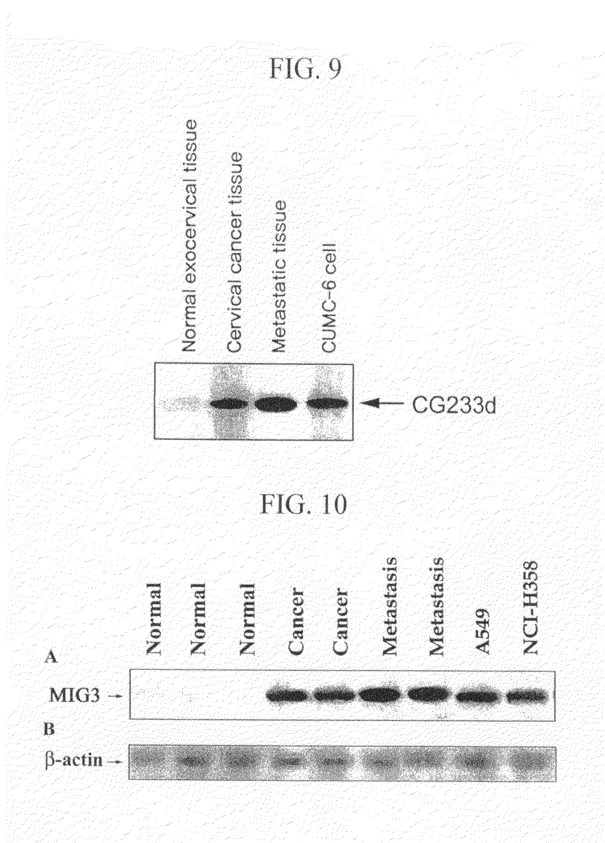

As shown in FIG. 9, it was revealed from the differential display (DD) that the gene was differentially expressed in the normal exocervical tissue, the metastatic lymph node tissue and the CUMC-6 cell. As seen in FIG. 9, the 327-bp cDNA fragment CG233 was expressed in the cervical cancer tissue, the metastatic lymph node tissue and the CUMC-6 cancer cell, but not expressed in the normal tissue.

### EXAMPLE 6

### cDNA Sequence Analysis of Full-length Protooncogene

6-1: MIG3

The ³²P-labeled L276-811 was used as the probe to screen a bacteriophage λgt11 human lung embryonic fibroblast cDNA library (Miki, T. et al., Gene 83: 137-146, 1989). The full-length MIG3 cDNA clone, in which the 2295-bp fragment was inserted into the pCEV-LAC vector, was obtained from the human lung embryonic fibroblast cDNA library, and then deposited with Accession No. AY239293 into the GenBank database of U.S. NIH on Feb. 19, 2003 (Publication Date: Dec. 31, 2004).

The MIG3 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG3 gene was ligated by T4 DNA ligase to obtain MIG3 plasmid DNA, and then E. coli DH5α was transformed with the ligated clone.

In the DNA sequence of SEQ ID NO: 1, it is estimated that a full-length open reading frame of the protooncogene of the present invention corresponds to nucleotide sequence positions from 89 to 709, and encodes a protein consisting of 206 amino acids of SEQ ID NO: 2.

6-2: MIG8

The ³²P-labeled CC231 was used as the probe to screen a bacteriophage λgt11 human lung embryonic fibroblast cDNA library (Miki, T. et al., Gene 83: 137-146, 1989). The full-length MIG8 cDNA clone, in which the 3737-bp fragment was inserted into the pCEV-LAC vector, was obtained from the human lung embryonic fibroblast cDNA library, and then deposited with Accession No. AY311389 into the GenBank database of U.S. NIH on Jun. 1, 2003 (Publication Date: Dec. 31, 2004).

The MIG8 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG8 gene was ligated by T4 DNA ligase to obtain MIG8 plasmid DNA, and then E. coli DH5α was transformed with the ligated clone.

The full-length DNA sequence of MIG18 consisting of 3737 bp was set forth in SEQ ID NO: 5.

In the DNA sequence of SEQ ID NO: 5, it is estimated that a full-length open reading frame of the protooncogene of the present invention corresponds to nucleotide sequence positions from 113 to 1627, and encodes a protein consisting of 504 amino acids of SEQ ID NO: 6.

6-3: MIG10

The ³²P-labeled L789 was used as the probe to screen a bacteriophage λgt11 human lung embryonic fibroblast cDNA library (Miki, T. et al., Gene 83: 137-146, 1989). The full-length MIG10 cDNA clone, in which the 1321-bp fragment was inserted into the PCEV-LAC vector, was obtained from the human lung embryonic fibroblast cDNA library, and then deposited with Accession No. AY423725 into the GenBank database of U.S. NIH on Sep. 26, 2003 (Publication Date: Dec. 31, 2004).

The MIG10 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG10 gene was ligated by T4 DNA ligase to obtain MIG10 plasmid DNA, and then E. coli DH5α was transformed with the ligated clone.

In the DNA sequence of SEQ ID NO: 9, it is estimated that a full-length open reading frame of the protooncogene of the present invention corresponds to nucleotide sequence positions from 23 to 1276, and encodes a protein consisting of 417 amino acids of SEQ ID NO: 10.

6-4: MIG13

The ³²P-labeled L986 was used as the probe to screen a bacteriophage λgt11 human lung embryonic fibroblast cDNA library (Miki, T. et al., Gene 83: 137-146, 1989). The full-length MIG13 cDNA clone, in which the 1019-bp fragment was inserted into the pCEV-LAC vector, was obtained from the human lung embryonic fibroblast cDNA library, and then deposited with Accession No. AY336090 into the GenBank database of U.S. NIH on Jul. 7, 2003 (Publication Date: Dec. 31, 2004).

The MIG13 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG13 gene was ligated by T4 DNA ligase to obtain MIG13 plasmid DNA, and then E. coli DH5α was transformed with the ligated clone.

In the DNA sequence of SEQ ID NO: 13, it is estimated that a full-length open reading frame of the protooncogene of the present invention corresponds to nucleotide sequence positions from 11 to 844, and encodes a protein consisting of 277 amino acids of SEQ ID NO: 14.

6-5: MIG14

The ³²P-labeled L1284 was used as the probe to screen a bacteriophage λgt11 human lung embryonic fibroblast cDNA library (Miki, T. et al., Gene 83: 137-146, 1989). The full-length MIG14 cDNA clone, in which the 1142-bp fragment was inserted into the pCEV-LAC vector, was obtained from the human lung embryonic fibroblast cDNA library, and then deposited with Accession No. AY336091 into the GenBank database of U.S. NIH on Jul. 4, 2003 (Publication Date: Dec. 31, 2004).

The MIG14 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG14 gene was ligated by T4 DNA ligase to obtain MIG14 plasmid DNA, and then E. coli DH5α was transformed with the ligated clone.

In the DNA sequence of SEQ ID NO: 17, it is estimated that a full-length open reading frame of the protooncogene of the present invention corresponds to nucleotide sequence positions from 67 to 1125, and encodes a protein consisting of 352 amino acids of SEQ ID NO: 18.

6-6: MIG18

The ³²P-labeled CA367 was used as the probe to screen a bacteriophage λgt11 human lung embryonic fibroblast cDNA library (Miki, T. et al., Gene 83: 137-146, 1989). The full-length MIG18 cDNA clone, in which the 3633-bp fragment was inserted into the pCEV-LAC vector, was obtained from the human lung embryonic fibroblast cDNA library, and then deposited with Accession No. AY423734 into the GenBank database of U.S. NIH on Sep. 30, 2003 (Publication Date: Dec. 31, 2004).

The MIG18 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG18 gene was ligated by T4 DNA ligase to obtain MIG18 plasmid DNA, and then E. coli DH5α was transformed with the ligated clone.

The full-length DNA sequence of MIG 18 consisting of 3633 bp was set forth in SEQ ID NO: 21.

In the DNA sequence of SEQ ID NO: 21, it is estimated that a full-length open reading frame of the protooncogene of the present invention corresponds to nucleotide sequence positions from 215 to 2212, and encodes a protein consisting of 665 amino acids of SEQ ID NO: 22.

6-7: MIG19

The ³²P-labeled CA335 was used as the probe to screen a bacteriophage λgt11 human lung embryonic fibroblast cDNA library (Miki, T. et al., Gene 83: 137-146, 1989). The full-length MIG19 cDNA clone, in which the 4639-bp fragment was inserted into the pCEV-LAC vector, was obtained from the human lung embryonic fibroblast cDNA library, and then deposited with Accession No. AY450308 into the GenBank database of U.S. NIH on Oct. 26, 2003 (Publication Date: Dec. 31, 2004).

The MIG19 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG19 gene was ligated by T4 DNA ligase to obtain MIG19 plasmid DNA, and then E. coli DH5α was transformed with the ligated clone.

The full-length DNA sequence of MIG19 consisting of 4639 bp was set forth in SEQ ID NO: 25.

In the DNA sequence of SEQ ID NO: 25, it is estimated that a full-length open reading frame of the protooncogene of the present invention corresponds to nucleotide sequence positions from 65 to 2965, and encodes a protein consisting of 966 amino acids of SEQ ID NO: 26.

6-8: MIG5

The ³²P-labeled CG263 was used as the probe to screen a bacteriophage λgt11 human lung embryonic fibroblast cDNA library (Miki, T. et al., Gene 83: 137-146, 1989). The full-length MIG5 cDNA clone, in which the 833-bp fragment was inserted into the pCEV-LAC vector, was obtained from the human lung embryonic fibroblast cDNA library, and then deposited with Accession No. AY279384 into the GenBank database of U.S. NIH on Apr. 19, 2003 (Publication Date: Dec. 31, 2004).

The MIG5 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG5 gene was ligated by T4 DNA ligase to obtain MIG5 plasmid DNA, and then E. coli DH5α was transformed with the ligated clone.

The full-length DNA sequence of MIG5 consisting of 833 bp was set forth in SEQ ID NO: 29.

In the DNA sequence of SEQ ID NO: 29, it is estimated that a full-length open reading frame of the protooncogene of the present invention corresponds to nucleotide sequence positions from 159 to 737, and encodes a protein consisting of 192 amino acids of SEQ ID NO: 30.

6-9: MIG7

The ³²P-labeled CG233 was used as the probe to screen a bacteriophage λgt11 human lung embryonic fibroblast cDNA library (Miki, T. et al., Gene 83: 137-146, 1989). The full-length MIG7 cDNA clone, in which the 2364-bp fragment was inserted into the pCEV-LAC vector, was obtained from the human lung embryonic fibroblast cDNA library, and then deposited with Accession No. AY305872 into the GenBank database of U.S. NIH on May 24, 2003 (Publication Date: Dec. 31, 2004).

The MIG7 clone inserted into the λpCEV vector was cleaved by the restriction enzyme NotI and isolated from the phage in the form of ampicillin-resistant pCEV-LAC phagemid vector (Miki, T. et al., Gene 83: 137-146, 1989).

The pCEV-LAC vector containing the MIG7 gene was ligated by T4 DNA ligase to obtain MIG7 plasmid DNA, and then E. coli DH5α was transformed with the ligated clone.

The full-length DNA sequence of MIG7 consisting of 2364 bp was set forth in SEQ ID NO: 33.

In the DNA sequence of SEQ ID NO: 33, it is estimated that a full-length open reading frame of the protooncogene of the present invention corresponds to nucleotide sequence positions from 1435 to 1665, and encodes a protein consisting of 76 amino acids of SEQ ID NO: 4.

### EXAMPLE 7

### Northern Blotting Analysis of Genes in Various Cells

7-1: MIG3, MIG10, MIG13 and MIG14

The total RNA samples were extracted from the normal lung tissue, the left lung cancer tissue, the metastatic lung cancer tissue metastasized from the left lung to the right lung, and the A549 and NCI-H358 (American Type Culture Collection; ATCC No. CRL-5807) lung cancer cell lines in the same manner as in Example 1.

In order to determine an expression level of each of the MIG3; MIG10; MIG13 and MIG14 genes, 20 μg of each of the total denatured RNA samples extracted from each of the tissues and the cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed full-length MIG cDNA probe prepared using the Rediprime II random prime labelling system ((Amersham, United Kingdom). The northern blotting analysis was repeated twice, and therefore the resultant blots were quantified with the densitometer and normalized with the β-actin.

FIG. 10(a) shows a northern blotting result to determine whether or not the MIG3 protooncogene is expressed in the normal lung tissue, the lung cancer tissue, the metastatic lung cancer tissue and the lung cancer cell lines (A549 and NCI-H358). As shown in FIG. 10(a), it was revealed that the expression level of the MIG3 protooncogene was significantly increased in the lung cancer tissue, the metastatic lung cancer tissue and the A549 and NCI-H358 lung cancer cell lines, but very low or not detected in the normal lung tissue. In FIG. 10(a), Lane “Normal” represents the normal lung tissue, Lane “Cancer” represents the lung cancer tissue, Lane “metastasis” represents the metastatic lung cancer tissue, and each of Lanes “A549” and “NCI-H358” represents the lung cancer cell line. FIG. 10(b) shows the northern blotting result indicating presence of mRNA transcript by hybridizing the same sample with β-actin probe.

FIG. 24(a) shows a northern blotting result to determine whether or not the MIG3 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 24(b) shows the northern blotting result indicating presence of mRNA transcript by hybridizing the same sample with β-actin probe. As shown in FIG. 24(a), it was revealed that the MIG3 mRNA transcript (approximately 4.0 kb) was very weakly expressed in the normal tissues.

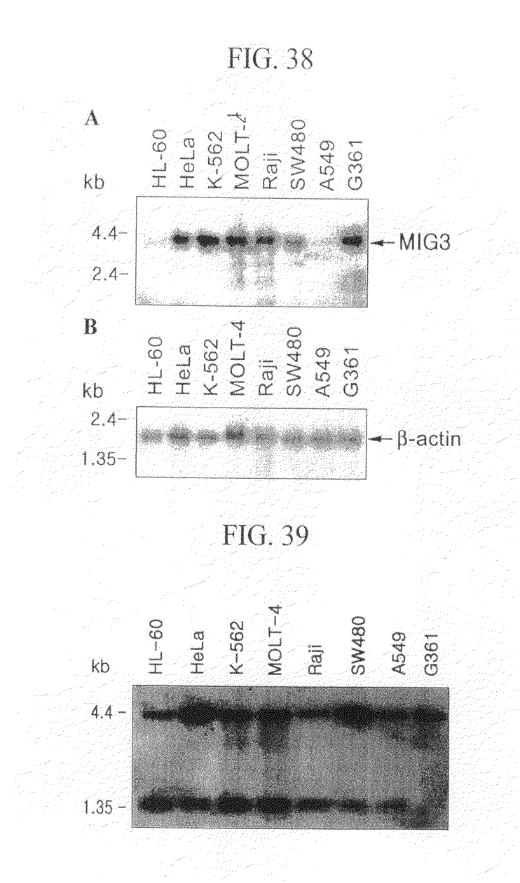

FIG. 38(a) shows a northern blotting result to determine whether or not the MIG3 protooncogene is expressed in the human cancer cell lines, for example HL-60, HeLa, K-562, MOLT-4, Raji, SW480, A549 and G361 (Clontech). FIG. 38(b) shows the northern blotting result indicating presence of mRNA transcript by hybridizing the same sample with β-actin probe. As shown in FIG. 38(a), it was revealed that the MIG3 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

FIG. 13(a) shows a northern blotting result to determine whether or not the MIG10 protooncogene is expressed in the normal lung tissue, the lung cancer tissue, the metastatic lung cancer tissue and the lung cancer cell lines (A549 and NCI-H358). As shown in FIG. 13(a), it was revealed that the expression level of the MIG10 protooncogene was significantly increased in the lung cancer tissue, the metastatic lung cancer tissue and the A549 and NCI-H358 lung cancer cell lines, but very low or not detected in the normal lung tissue. In FIG. 13(a), Lane “Normal” represents the normal lung tissue, Lane “Cancer” represents the lung cancer tissue, Lane “metastasis” represents the metastatic lung cancer tissue, and each of Lanes “A549” and “NCI-H358” represents the lung cancer cell line. FIG. 13(b) shows the northern blotting result indicating presence of mRNA transcript by hybridizing the same sample with β-actin probe.

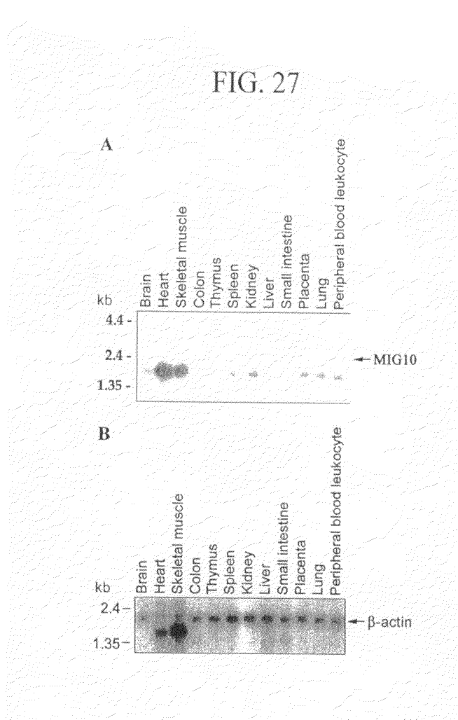

FIG. 27(a) shows a northern blotting result to determine whether or not the MIG10 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 27(b) shows the northern blotting result indicating presence of mRNA transcript by hybridizing the same sample with β-actin probe. As shown in FIG. 27(a), it was revealed that the MIG10 mRNA transcript (approximately 2.0 kb) was very weakly expressed in the normal tissues.

FIG. 41(a) shows a northern blotting result to determine whether or not the MIG10 protooncogene is expressed in the human cancer cell lines, for example HL-60, HeLa, K-562, MOLT-4, Raji, SW480, A549 and G361 (Clontech). FIG. 41(b) shows the northern blotting result indicating presence of mRNA transcript by hybridizing the same sample with β-actin probe. As shown in FIG. 41(a), it was revealed that the MIG10 protooncogene was very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. It was also seen that mRNA transcript of approximately 2.4 kb was expressed in addition to the 2.0-kb mRNA transcript.

FIG. 14(a) shows a northern blotting result to determine whether or not the MIG 13 protooncogene is expressed in the normal lung tissue, the lung cancer tissue, the metastatic lung cancer tissue and the lung cancer cell lines (A549 and NCI-H358). As shown in FIG. 14(a), it was revealed that the expression level of the MIG13 protooncogene was significantly increased in the lung cancer tissue, the metastatic lung cancer tissue and the A549 and NCI-H358 lung cancer cell lines, but very low or not detected in the normal lung tissue. In FIG. 14(a), Lane “Normal” represents the normal lung tissue, Lane “Cancer” represents the lung cancer tissue, Lane “metastasis” represents the metastatic lung cancer tissue, and each of Lanes “A549” and “NCI-H358” represents the lung cancer cell line. FIG. 14(b) shows the northern blotting result indicating presence of mRNA transcript by hybridizing the same sample with β-actin probe.

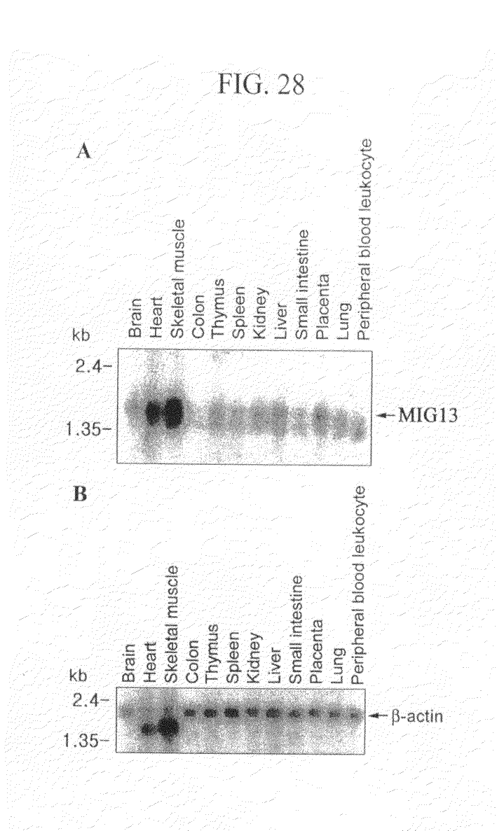

FIG. 28(a) shows a northern blotting result to determine whether or not the MIG13 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 28(b) shows the northern blotting result indicating presence of mRNA transcript by hybridizing the same sample with β-actin probe. As shown in FIG. 28(a), it was revealed that the MIG13 mRNA transcripts (a dominant transcript of approximately 1.7 kb and a transcript of 1.4 kb) were very weakly expressed or not detected in the normal tissues.

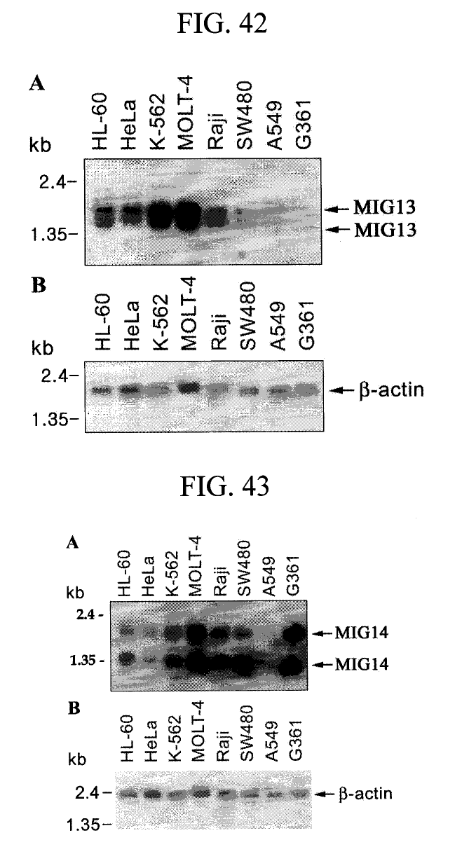

FIG. 42(a) shows a northern blotting result to determine whether or not the MIG13 protooncogene is expressed in the human cancer cell lines, for example HL-60, HeLa, K-562, MOLT-4, Raji, SW480, A549 and G361 (Clontech). FIG. 42(b) shows the northern blotting result indicating presence of mRNA transcript by hybridizing the same sample with β-actin probe. As shown in FIG. 42(a), it was revealed that the MIG14 mRNA transcripts (a dominant transcript of approximately 1.7 kb and a transcript of 1.4 kb) were very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

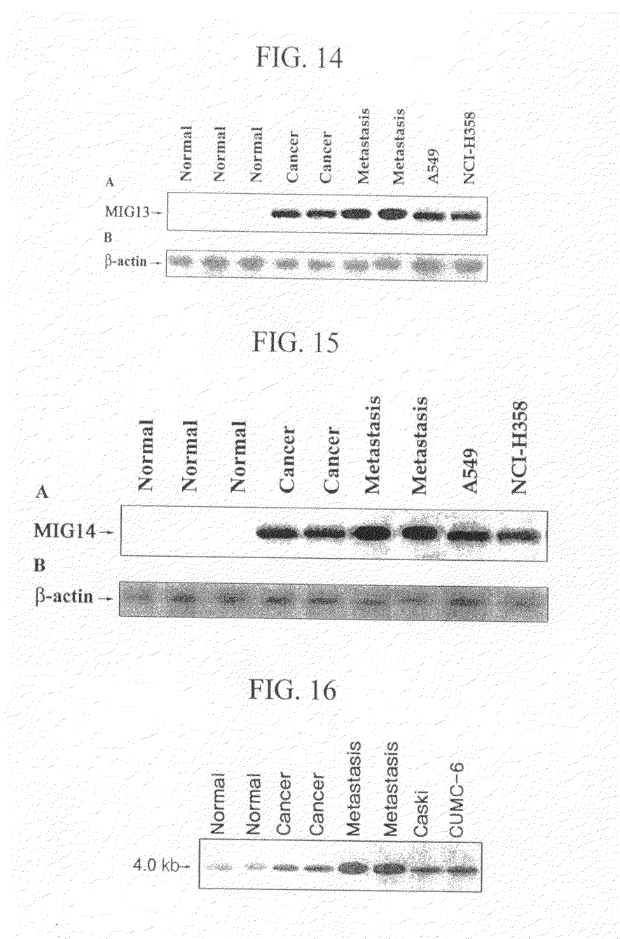

FIG. 15(a) shows a northern blotting result to determine whether or not the MIG14 protooncogene is expressed in the normal lung tissue, the lung cancer tissue, the metastatic lung cancer tissue and the lung cancer cell lines (A549 and NCI-H358). As shown in FIG. 15(a), it was revealed that the expression level of the MIG14 protooncogene was significantly increased in the lung cancer tissue, the metastatic lung cancer tissue and the A549 and NCI-H358 lung cancer cell lines, but very low or not detected in the normal lung tissue. In FIG. 15, Lane “Normal” represents the normal lung tissue, Lane “Cancer” represents the lung cancer tissue, Lane “metastasis” represents the metastatic lung cancer tissue, and each of Lanes “A549” and “NCI-H358” represents the lung cancer cell line. FIG. 15(b) shows the northern blotting result indicating presence of mRNA transcript by hybridizing the same sample with β-actin probe.

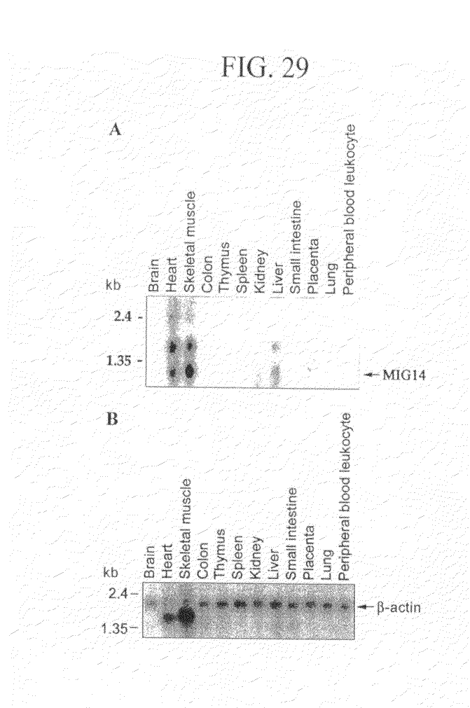

FIG. 29(a) shows a northern blotting result to determine whether or not the MIG14 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 29(b) shows the northern blotting result indicating presence of mRNA transcript by hybridizing the same sample with β-actin probe. As shown in FIG. 29(a), it was revealed that the MIG14 mRNA transcripts (a dominant transcript of approximately 1.3 kb and a transcript of 2 kb) were very weakly expressed or not detected in the normal tissues.

FIG. 43(a) shows a northern blotting result to determine whether or not the MIG14 protooncogene is expressed in the human cancer cell lines, for example HL-60, HeLa, K-562, MOLT-4, Raji, SW480, A549 and G361 (Clontech). FIG. 43(b) shows the northern blotting result indicating presence of mRNA transcript by hybridizing the same sample with β-actin probe. As shown in FIG. 43(a), it was revealed that the MIG14 mRNA transcripts (a dominant transcript of approximately 1.3 kb and a transcript of 2 kb) were very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361.

7-2: MIG8, MIG18, MIG19, MIG5 and MIG7

The total RNA samples were extracted from the normal exocervical tissue, the cervical cancer tissue, the metastatic cervical lymph node tissue and the cervical cancer cell lines CaSki (ATCC CRL 1550) and CUMC-6 in the same manner as in Example 1.

In order to determine an expression level of each of the MIG8; MIG18; MIG19; MIG5 and MIG7 genes, 20 μg of each of the total denatured RNA samples extracted from each of the tissues and cell lines was electrophoresized in an 1% formaldehyde agarose gel, and then the resultant agarose gel were transferred to a nylon membrane ((Boehringer-Mannheim, Germany). The blot was then hybridized with the ³²P-labeled and randomly primed full-length MIG cDNA probe prepared using the Rediprime II random prime labelling system ((Amersham, United Kingdom). The northern blotting analysis was repeated twice, and therefore the resultant blots were quantified with the densitometer and normalized with the β-actin.

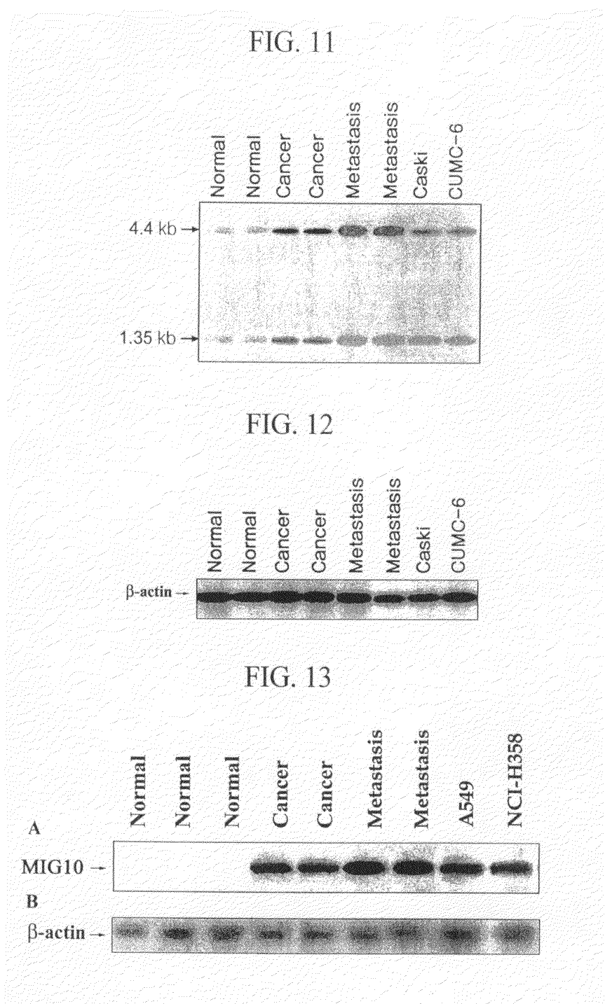

FIG. 11 shows a northern blotting result to determine whether or not the MIG8 protooncogene is expressed in the normal exocervical tissue, the cervical cancer tissue, the metastatic cervical lymph node tissue and the cervical cancer cell lines (CaSki and CUMC-6). As shown in FIG. 11, it was revealed that the expression level of the MIG8 protooncogene was increased in the cervical cancer tissue and the cervical cancer cell lines CaSki and CUMC-6, that is, a dominant MIG8 mRNA transcript of approximately 4.0 kb and an MIG8 mRNA transcript of approximately 1.3 kb were overexpressed, and the MIG8 protooncogene was the most highly expressed especially in the metastatic cervical lymph node tissue, but very low expressed in the normal tissue. In FIG. 11, Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 12 shows the northern blotting result indicating presence of mRNA transcript by hybridizing the same sample with β-actin probe.

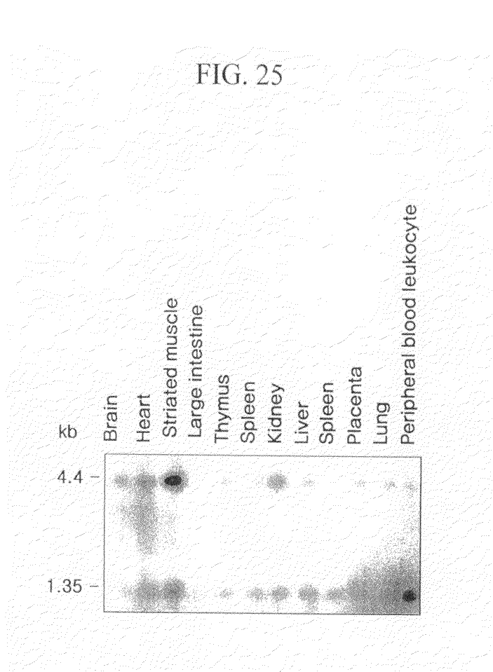

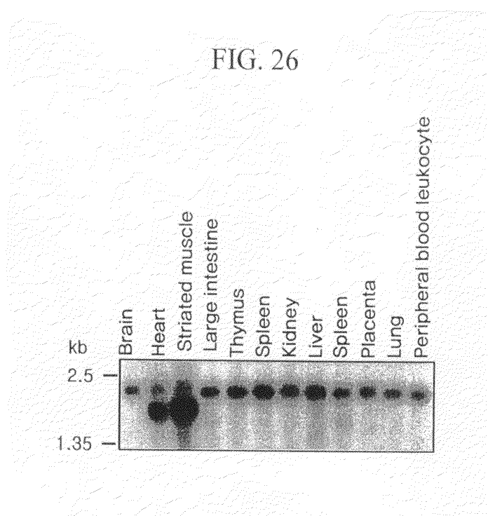

FIG. 25 shows a northern blotting result to determine whether or not the MIG8 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 26 shows the northern blotting result indicating presence of mRNA transcript by hybridizing the same sample with β-actin probe. As shown in FIG. 25, it was revealed that the MIG8 mRNA transcripts (a dominant MIG8 mRNA transcript of approximately 4.0 kb and an MIG8 mRNA transcript of approximately 1.3 kb) were weakly expressed in the normal tissues such as brain, heart, striated muscle, large intestines, thymus, spleen, kidneys, liver, small intestines, placenta, lungs and peripheral blood leukocyte.

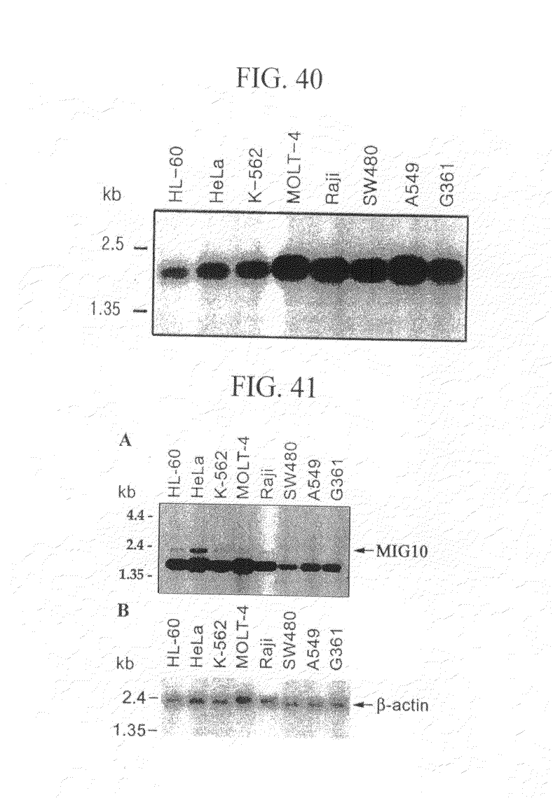

FIG. 39 shows a northern blotting result to determine whether or not the MIG8 protooncogene is expressed in the human cancer cell lines, for example HL-60, HeLa, K-562, MOLT-4, Raji, SW480, A549 and G361 (Clontech). FIG. 40 shows the northern blotting result indicating presence of mRNA transcript by hybridizing the same sample with β-actin probe. As shown in FIG. 39, it was revealed that the MIG8 mRNA transcripts (a dominant MIG8 mRNA transcript of approximately 4.0 kb and an MIG8 mRNA transcript of approximately 1.3 kb) were very highly expressed in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. But, the MIG8 mRNA transcript of approximately 1.3 kb was not expressed in the skin cancer cell line G361.

FIG. 16 shows a northern blotting result to determine whether or not the MIG18 protooncogene is expressed in the normal exocervical tissue, the cervical cancer tissue, the metastatic cervical lymph node tissue and the cervical cancer cell lines (CaSki and CUMC-6). As shown in FIG. 16, it was revealed that the expression level of the MIG18 protooncogene was increased in the cervical cancer tissue and the cervical cancer cell lines CaSki and CUMC-6, and the MIG18 protooncogene was the most highly expressed especially in the metastatic cervical lymph node tissue, but very low expressed in the normal tissue. In FIGS. 16 and 17, Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 17 shows the northern blotting result indicating presence of mRNA transcript by hybridizing the same sample with β-actin probe.

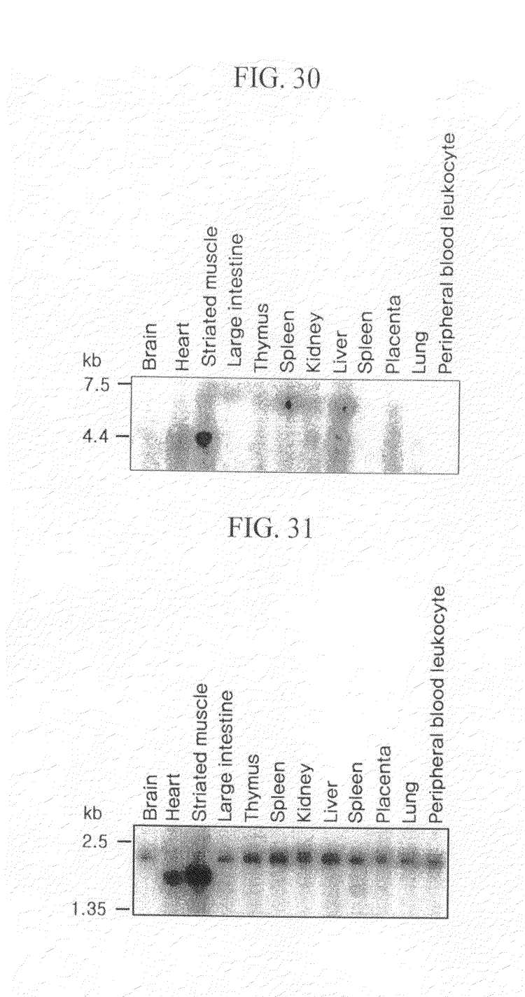

FIG. 30 shows a northern blotting result to determine whether or not the MIG18 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 31 shows the northern blotting result indicating presence of mRNA transcript by hybridizing the same sample with β-actin probe. As shown in FIG. 30, it was revealed that the MIG18 mRNA transcript (approximately 4.0 kb) was weakly expressed in the normal tissues such as heart, muscle and liver.

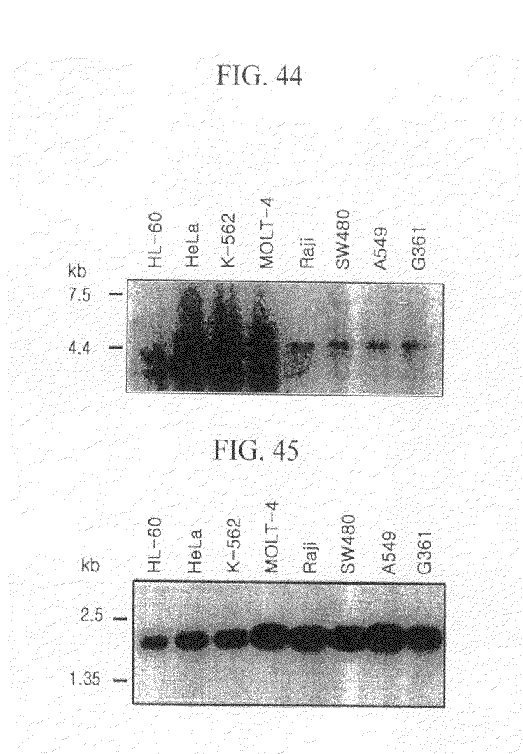

FIG. 44 shows a northern blotting result to determine whether or not the MIG18 protooncogene is expressed in the human cancer cell lines, for example HL-60, HeLa, K-562, MOLT-4, Raji, SW480, A549 and G361 (Clontech). FIG. 45 shows the northern blotting result indicating presence of mRNA transcript by hybridizing the same sample with β-actin probe. As shown in FIG. 44, it was revealed that the MIG18 mRNA transcript was very highly expressed in the HeLa uterine cancer cell line and the chronic myelogenous leukemia cell line K-562, and also expressed at a increased level in the promyelocyte leukemia cell line HL-60, the lymphoblastic leukaemia cell line MOLT-4, the Burkitt lymphoma cell line Raji, the colon cancer cell line SW480, the lung cancer cell line A549 and the skin cancer cell line G361.

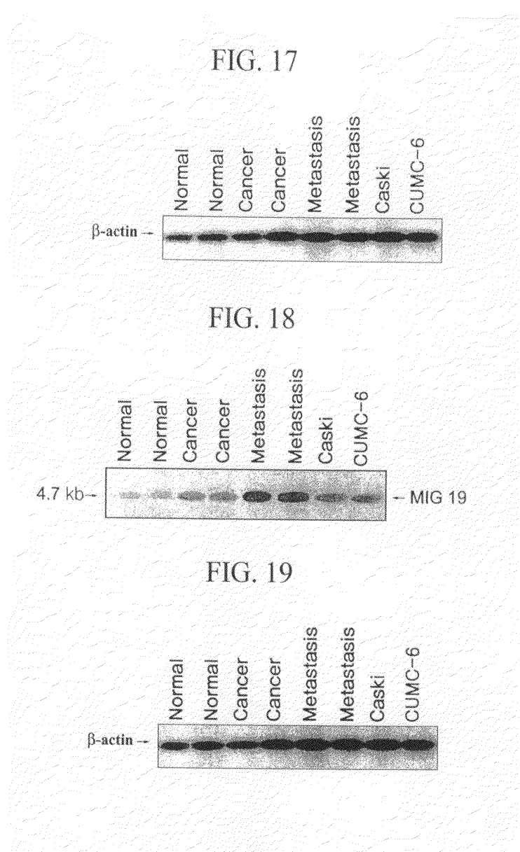

FIG. 18 shows a northern blotting result to determine whether or not the MIG19 protooncogene is expressed in the normal exocervical tissue, the cervical cancer tissue, the metastatic cervical lymph node tissue and the cervical cancer cell lines (CaSki and CUMC-6). As shown in FIG. 18, it was revealed that the expression level of the MIG19 protooncogene was increased in the cervical cancer tissue and the cervical cancer cell lines CaSki and CUMC-6, that is, dominant MIG19 mRNA transcript of approximately 4.7 kb was overexpressed, and the MIG19 protooncogene was the most highly expressed especially in the metastatic cervical lymph node tissue, but very low expressed in the normal tissue. In FIGS. 18 and 19, Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 19 shows the northern blotting result indicating presence of mRNA transcript by hybridizing the same sample with β-actin probe.

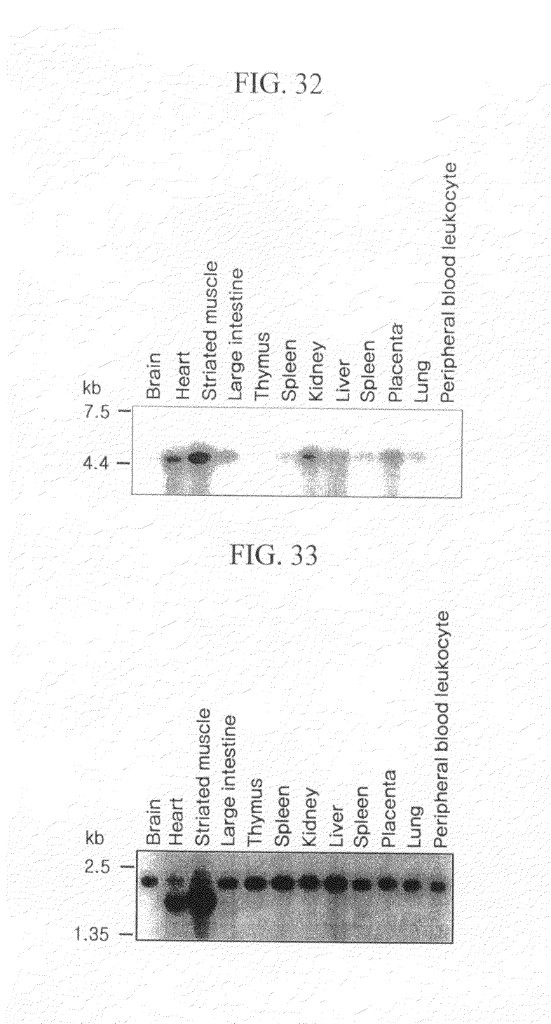

FIG. 32 shows a northern blotting result to determine whether or not the MIG19 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 33 shows the northern blotting result indicating presence of mRNA transcript by hybridizing the same sample with β-actin probe. As shown in FIG. 32, it was revealed that the MIG19 mRNA transcript (a dominant mRNA transcript of approximately 4.7 kb) was weakly expressed or not detected in the normal tissues such as brain, heart, striated muscle, large intestines, thymus, spleen, kidneys, liver, small intestines, placenta, lungs and peripheral blood leukocyte.

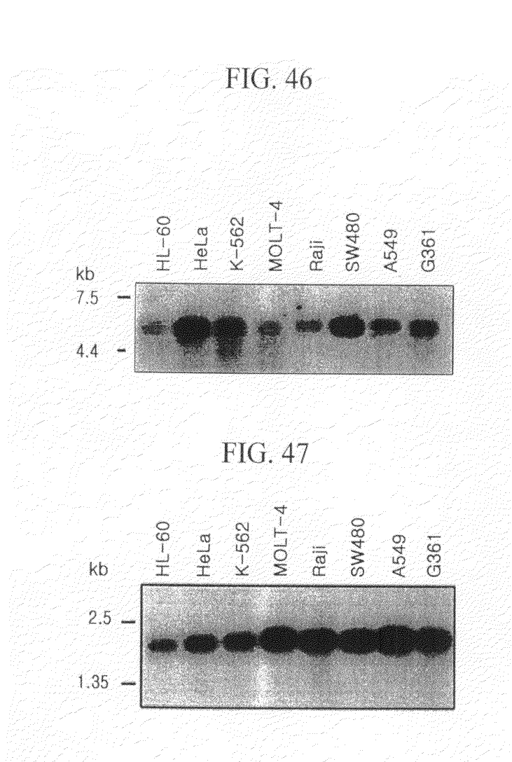

FIG. 46 shows a northern blotting result to determine whether or not the MIG19 protooncogene is expressed in the human cancer cell lines, for example HL-60, HeLa, K-562, MOLT-4, Raji, SW480, A549 and G361 (Clontech). FIG. 47 shows the northern blotting result indicating presence of mRNA transcript by hybridizing the same sample with β-actin probe. As shown in FIG. 46, it was revealed that the MIG19 mRNA transcripts (a dominant mRNA transcript of approximately 4.7 kb) were expressed at a very increased level in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. But, the MIG8 mRNA transcript of approximately 1.3 kb was not expressed in the skin cancer cell line G361.

FIG. 20 shows a northern blotting result to determine whether or not the MIG5 protooncogene is expressed in the normal exocervical tissue, the cervical cancer tissue, the metastatic cervical lymph node tissue and the cervical cancer cell lines (CaSki and CUMC-6).

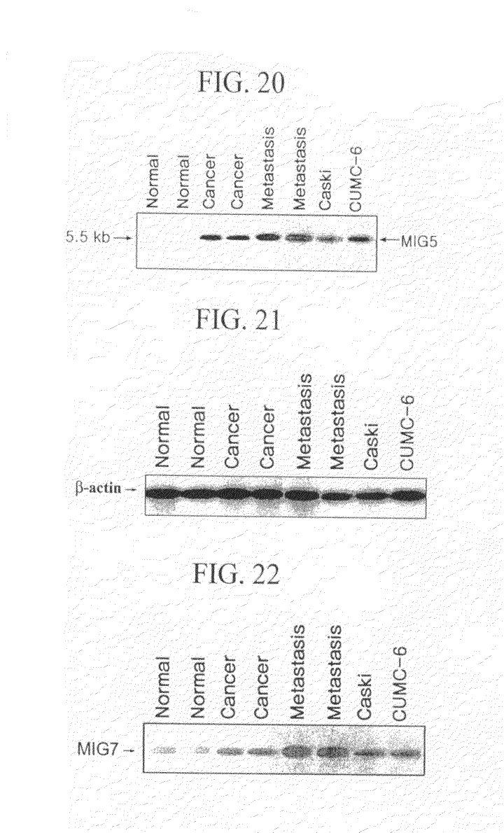

As shown in FIG. 20, it was revealed that the expression level of the MIG5 protooncogene was increased in the cervical cancer tissue and the cervical cancer cell lines CaSki and CUMC-6, that is, a dominant MIG5 mRNA transcript of approximately 5.5 kb were overexpressed, and the MIG5 protooncogene was the most highly expressed especially in the metastatic cervical lymph node tissue, but not expressed in the normal tissue. In FIGS. 20 and 21, Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 21 shows the northern blotting result indicating presence of mRNA transcript by hybridizing the same sample with β-actin probe.

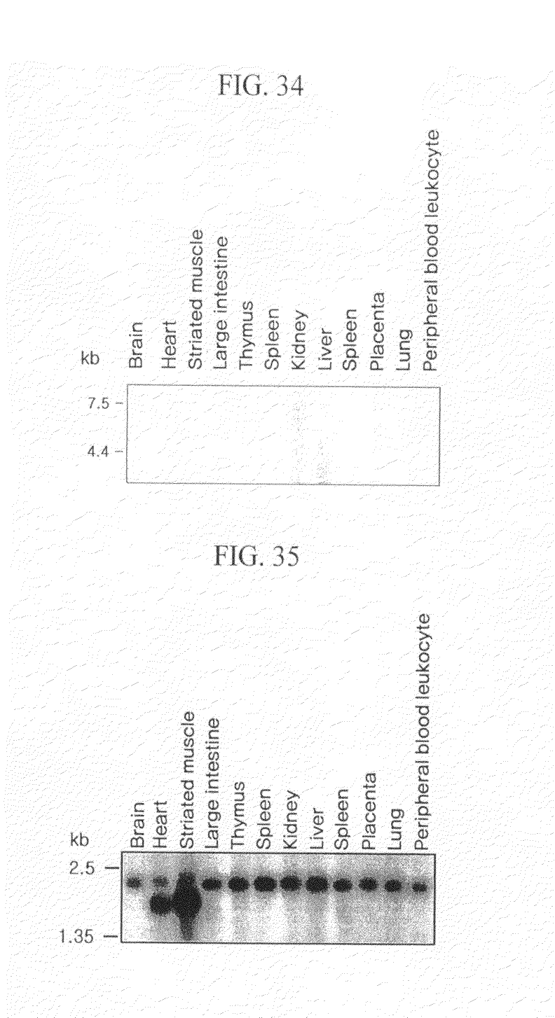

FIG. 34 shows a northern blotting result to determine whether or not the MIG5 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 35 shows the northern blotting result indicating presence of mRNA transcript by hybridizing the same sample with β-actin probe. As shown in FIG. 34, it was revealed that the MIG5 mRNA transcript (a dominant mRNA transcript of approximately 5.5 kb) was not expressed in the normal tissues such as brain, heart, striated muscle, large intestines, thymus, spleen, kidneys, liver, small intestines, placenta, lungs and peripheral blood leukocyte.

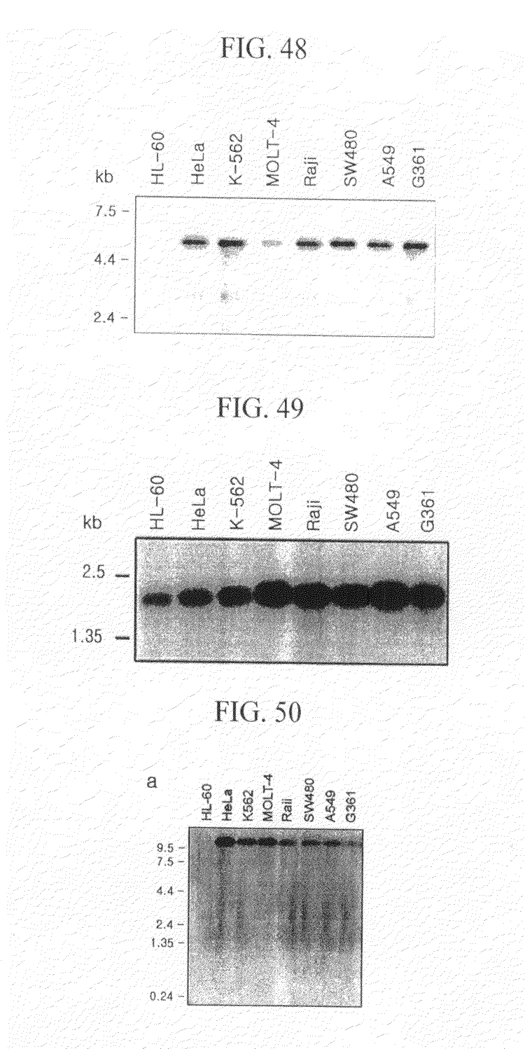

FIG. 48 shows a northern blotting result to determine whether or not the MIG5 protooncogene is expressed in the human cancer cell lines, for example HL-60, HeLa, K-562, MOLT-4, Raji, SW480, A549 and G361 (Clontech). FIG. 49 shows the northern blotting result indicating presence of mRNA transcript by hybridizing the same sample with β-actin probe. As shown in FIG. 48, it was revealed that the MIG5 mRNA transcript (a dominant mRNA transcript of approximately 5.5 kb) was expressed at a very increased level in the promyelocyte leukemia cell line HL-60, the HeLa uterine cancer cell line, the chronic myelogenous leukemia cell line K-562, the lymphoblastic leukaemia cell line MOLT-4, the Burkitt lymphoma cell line Raji, the colon cancer cell line SW480, the lung cancer cell line A549 and the skin cancer cell line G361. But, the MIG8 mRNA transcript of approximately 1.3 kb was not expressed in the skin cancer cell line G361.

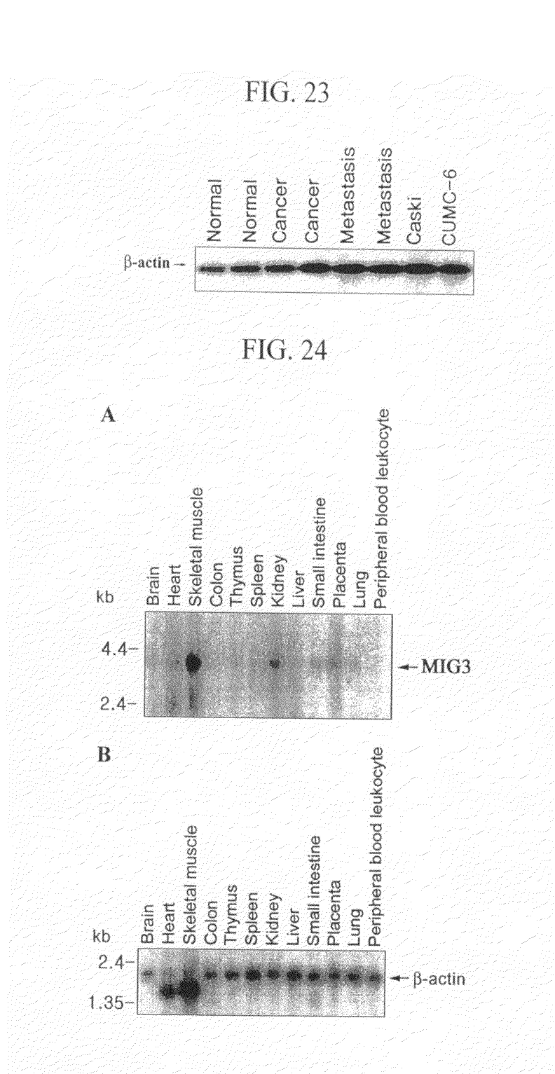

FIG. 22 shows a northern blotting result to determine whether or not the MIG19 protooncogene is expressed in the normal exocervical tissue, the cervical cancer tissue, the metastatic cervical lymph node tissue and the cervical cancer cell lines (CaSki and CUMC-6). As shown in FIG. 22, it was revealed that the expression level of the MIG7 protooncogene was increased in the cervical cancer tissue and the cervical cancer cell lines CaSki and CUMC-6, that is, dominant MIG7 mRNA transcript of approximately 10 kb was overexpressed, and the MIG7 protooncogene was the most highly expressed especially in the metastatic cervical lymph node tissue, but very low expressed in the normal tissue. In FIGS. 22 and 23, Lane “Normal” represents the normal exocervical tissue, Lane “Cancer” represents the cervical cancer tissue, Lane “metastasis” represents the metastatic cervical lymph node tissue, and each of Lanes “CaSki” and “CUMC-6” represents the uterine cancer cell line. FIG. 23 shows the northern blotting result indicating presence of mRNA transcript by hybridizing the same sample with β-actin probe.

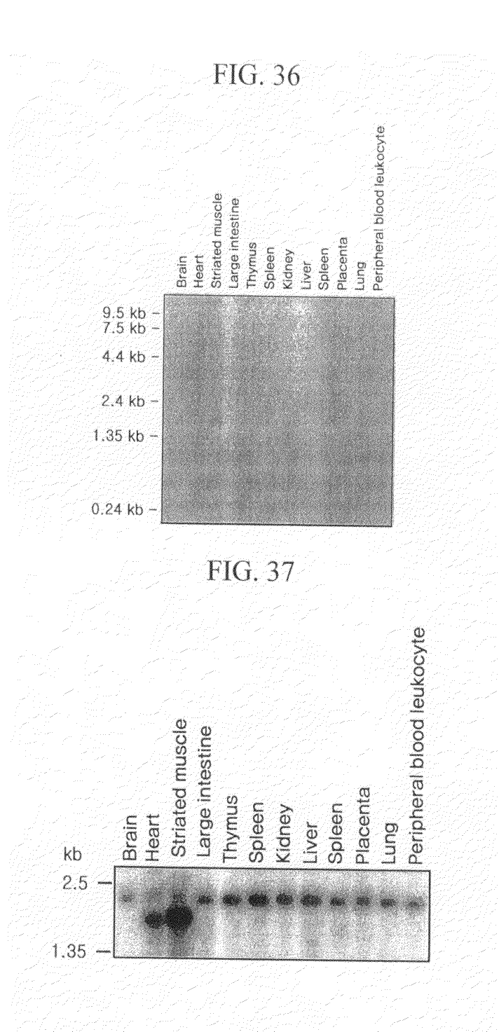

FIG. 36 shows a northern blotting result to determine whether or not the MIG19 protooncogene is expressed in the normal human 12-lane multiple tissues (Clontech), for example brain, heart, striated muscle, large intestines, thymus, spleen, kidneys, liver, small intestines, placenta, lungs and peripheral blood leukocyte tissues. FIG. 37 shows the northern blotting result indicating presence of mRNA transcript by hybridizing the same sample with β-actin probe. As shown in FIG. 36, it was revealed that the MIG7 mRNA transcript (dominant mRNA transcript of approximately 10 kb) was weakly expressed or not detected in the normal tissues such as brain, heart, striated muscle, large intestines, thymus, spleen, kidneys, liver, small intestines, placenta, lungs and peripheral blood leukocyte.

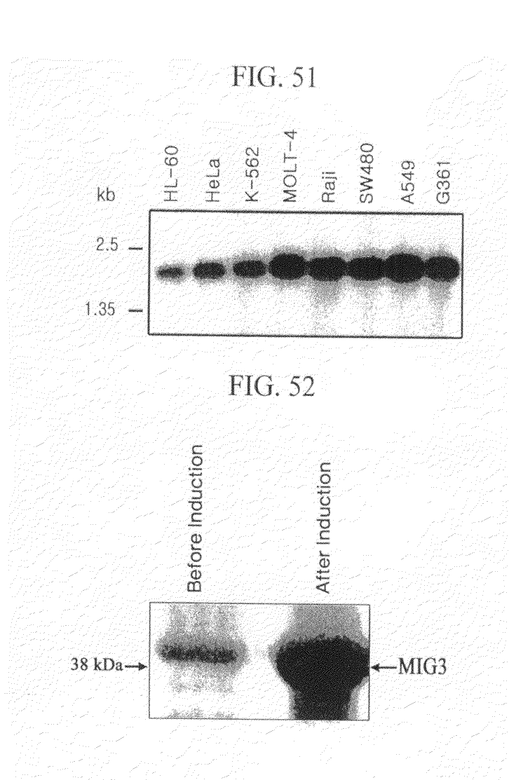

FIG. 50 shows a northern blotting result to determine whether or not the MIG7 protooncogene is expressed in the human cancer cell lines, for example HL-60, HeLa, K-562, MOLT-4, Raji, SW480, A549 and G361 (Clontech). FIG. 51 shows the northern blotting result indicating presence of mRNA transcript by hybridizing the same sample with β-actin probe. As shown in FIG. 50, it was revealed that the MIG7 mRNA transcript (a dominant mRNA transcript of approximately 10 kb) was expressed at a very increased level in the HeLa uterine cancer cell line, the chronic myelogenous leukemia cell line K-562, the lymphoblastic leukaemia cell line MOLT-4, the Burkitt lymphoma cell line Raji, the colon cancer cell line SW480 and the lung cancer cell line A549.

### EXAMPLE 8

### Size Determination of Protein Expressed after Transforming E. coli with Protooncogene

Each of the full-length MIG protooncogenes such as MIG3 of SEQ ID NO: 1; MIG8 of SEQ ID NO: 5; MIG10 of SEQ ID NO: 9; MIG13 of SEQ ID NO: 13; MIG14 of SEQ ID NO: 17; MIG18 of SEQ ID NO: 21; MIG 19 of SEQ ID NO: 25; MIG 5 of SEQ ID NO: 29; and MIG 7 of SEQ ID NO: 33 was inserted into a multi-cloning site of the pBAD/thio-Topo vector (Invitrogen, U.S.), and then E. coli Top10 (Invitrogen, U.S.) was transformed with each of the resultant pBAD/thio-Topo/MIG vectors. The expression proteins HT-Thioredoxin is inserted into a upstream region of the multi-cloning site of the pBAD/thio-Topo vector. Each of the transformed E. coli strains was incubated in LB broth while shaking, and then each of the resultant cultures was diluted at a ratio of 1/100 and incubated for 3 hours. 0.5 mM L-arabinose (Sigma) was added thereto to facilitate production of proteins.

The E. coli cells was sonicated in the cultures before/after the L-arabinose induction, and then the sonicated homogenates were subject to 12% sodium dodecyl sulfate-polyacrylamide gel electrophoresis (SDS-PAGE).

FIG. 52 shows a SDS-PAGE result to determine an expression pattern of the proteins in the E. coli Top10 strain transformed with the pBAD/thio-Topo/MIG3 vector, wherein a band of a fusion protein having a molecular weight of approximately 38 kDa was clearly observed after L-arabinose induction. The 38-kDa fusion protein includes the HT-thioredoxin protein having a molecular weight of approximately 15 kDa and the MIG3 protein having a molecular weight of approximately 23 kDa, each protein inserted into the pBAD/thio-Topo/MIG3 vector.

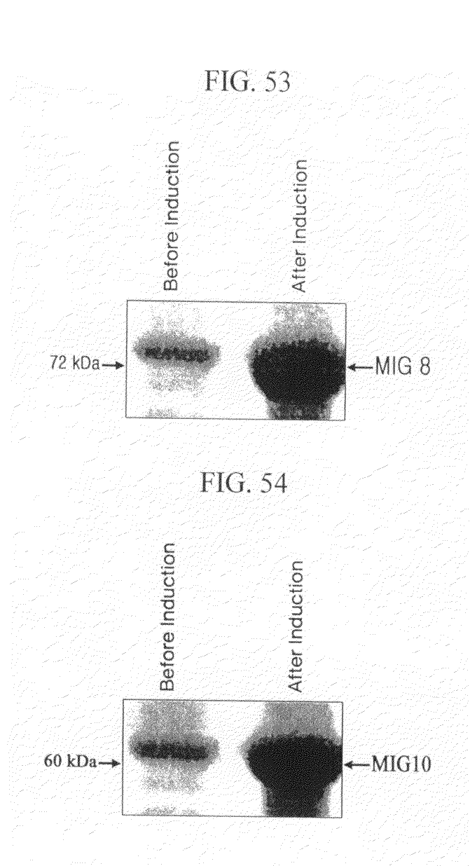

FIG. 53 shows a SDS-PAGE result to determine an expression pattern of the proteins in the E. coli Top10 strain transformed with the pBAD/thio-Topo/MIG8 vector, wherein a band of a fusion protein having a molecular weight of approximately 72 kDa was clearly observed after L-arabinose induction. The 72-kDa fusion protein includes the HT-thioredoxin protein having a molecular weight of approximately 15 kDa and the MIG8 protein having a molecular weight of approximately 57 kDa, each protein inserted into the pBAD/thio-Topo/MIG8 vector.

FIG. 54 shows a SDS-PAGE result to determine an expression pattern of the proteins in the E. coli Top10 strain transformed with the pBAD/thio-Topo/MIG10 vector, wherein a band of a fusion protein having a molecular weight of approximately 60 kDa was clearly observed after L-arabinose induction. The 60-kDa fusion protein includes the HT-thioredoxin protein having a molecular weight of approximately 15 kDa and the MIG10 protein having a molecular weight of approximately 45 kDa, each protein inserted into the pBAD/thio-Topo/MIG10 vector.

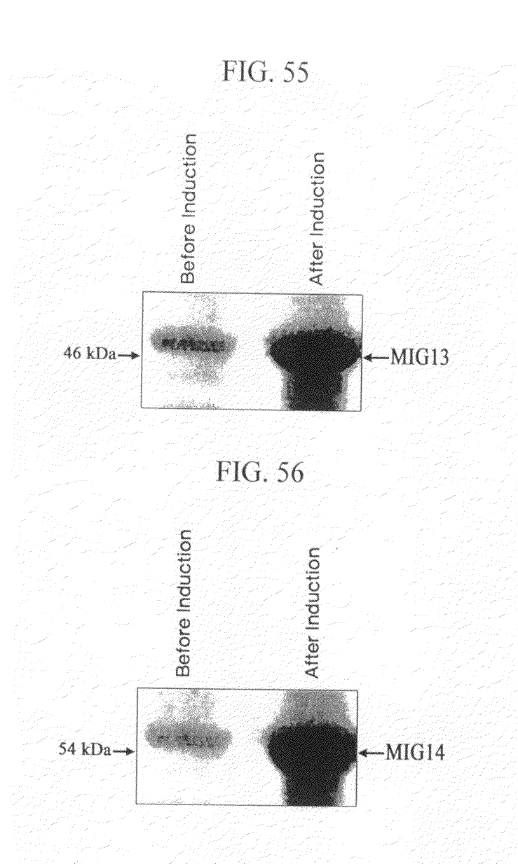

FIG. 55 shows a SDS-PAGE result to determine an expression pattern of the proteins in the E. coli Top10 strain transformed with the pBAD/thio-Topo/MIG13 vector, wherein a band of a fusion protein having a molecular weight of approximately 46 kDa was clearly observed after L-arabinose induction. The 46-kDa fusion protein includes the HT-thioredoxin protein having a molecular weight of approximately 15 kDa and the MIG13 protein having a molecular weight of approximately 31 kDa, each protein inserted into the pBAD/thio-Topo/MIG13 vector.

FIG. 56 shows a SDS-PAGE result to determine an expression pattern of the proteins in the E. coli Top10 strain transformed with the pBAD/thio-Topo/MIG14 vector, wherein a band of a fusion protein having a molecular weight of approximately 54 kDa was clearly observed after L-arabinose induction. The 54-kDa fusion protein includes the HT-thioredoxin protein having a molecular weight of approximately 15 kDa and the MIG14 protein having a molecular weight of approximately 39 kDa, each protein inserted into the pBAD/thio-Topo/MIG14 vector.

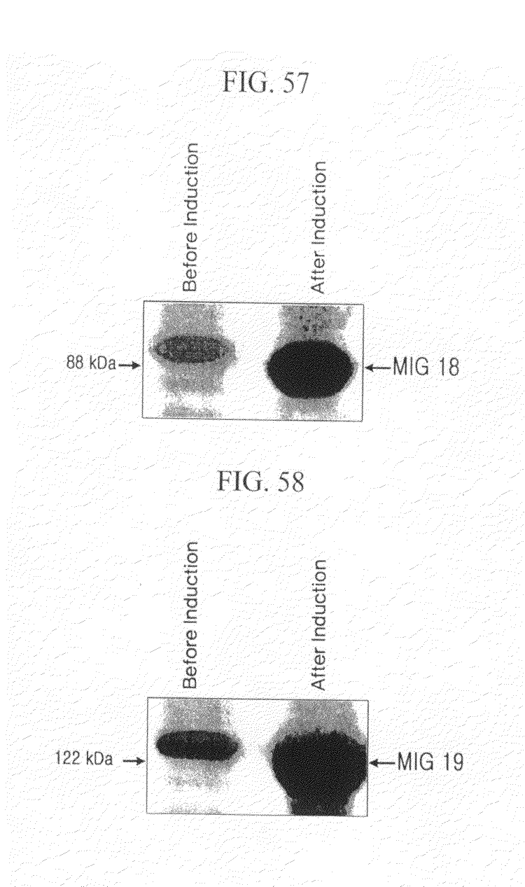

FIG. 57 shows a SDS-PAGE result to determine an expression pattern of the proteins in the E. coli Top10 strain transformed with the pBAD/thio-Topo/MIG18 vector, wherein a band of a fusion protein having a molecular weight of approximately 88 kDa was clearly observed after L-arabinose induction. The 88-kDa fusion protein includes the HT-thioredoxin protein having a molecular weight of approximately 15 kDa and the MIG18 protein having a molecular weight of approximately 73 kDa, each protein inserted into the pBAD/thio-Topo/MIG18 vector.

FIG. 58 shows a SDS-PAGE result to determine an expression pattern of the proteins in the E. coli Top10 strain transformed with the pBAD/thio-Topo/MIG19 vector, wherein a band of a fusion protein having a molecular weight of approximately 122 kDa was clearly observed after L-arabinose induction. The 122-kDa fusion protein includes the HT-thioredoxin protein having a molecular weight of approximately 15 kDa and the MIG19 protein having a molecular weight of approximately 107 kDa, each protein inserted into the pBAD/thio-Topo/MIG19 vector.

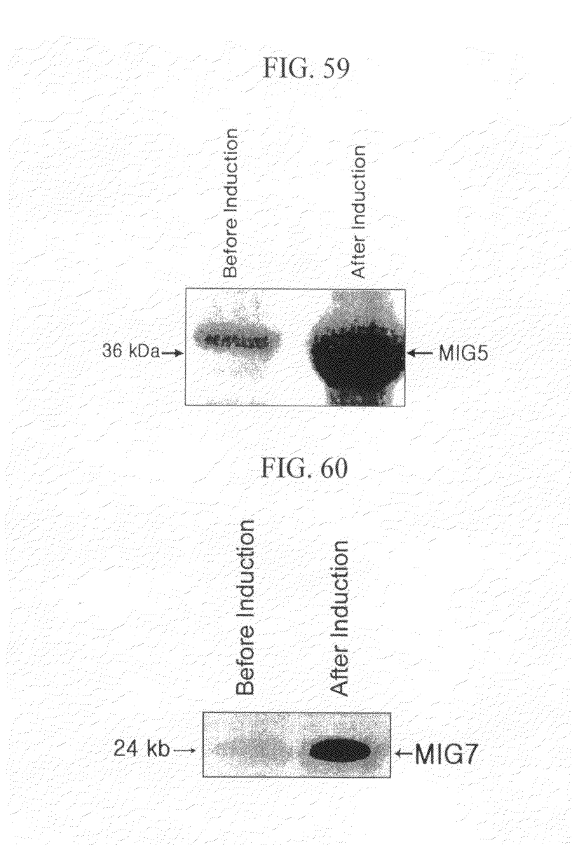

FIG. 59 shows a SDS-PAGE result to determine an expression pattern of the proteins in the E. coli Top10 strain transformed with the pBAD/thio-Topo/MIG5 vector, wherein a band of a fusion protein having a molecular weight of approximately 36 kDa was clearly observed after L-arabinose induction. The 36-kDa fusion protein includes the HT-thioredoxin protein having a molecular weight of approximately 15 kDa and the MIG5 protein having a molecular weight of approximately 21 kDa, each protein inserted into the pBAD/thio-Topo/MIG5 vector.

FIG. 60 shows a SDS-PAGE result to determine an expression pattern of the proteins in the E. coli Top10 strain transformed with the pBAD/thio-Topo/MIG7 vector, wherein a band of a fusion protein having a molecular weight of approximately 24 kDa was clearly observed after L-arabinose induction. The 24-kDa fusion protein includes the HT-thioredoxin protein having a molecular weight of approximately 15 kDa and the MIG7 protein having a molecular weight of approximately 9 kDa, each protein inserted into the pBAD/thio-Topo/MIG7 vector.

### INDUSTRIAL APPLICABILITY

As described above, the protooncogenes of the present invention, which are novel genes that takes part in human carcinogenesis and simultaneously has an ability to induce cancer metastasis, may be effectively used for diagnosing the cancers, including lung cancer, leukemia, uterine cancer, lymphoma, colon cancer, skin cancer, etc., as well as producing transformed animals, etc.

1. A human protooncoprotein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; and SEQ ID NO: 34.

2. A human protooncogene having a DNA sequence selected from the group consisting of a DNA sequence corresponding to nucleotide sequence positions from 89 to 709 of SEQ ID NO: 1; a DNA sequence corresponding to nucleotide sequence positions from 113 to 1627 of SEQ ID NO: 5; a DNA sequence corresponding to nucleotide sequence positions from 23 to 1276 of SEQ ID NO: 9; a DNA sequence corresponding to nucleotide sequence positions from 11 to 844 of SEQ ID NO: 13; a DNA sequence corresponding to nucleotide sequence positions from 67 to 1125 of SEQ ID NO: 17; a DNA sequence corresponding to nucleotide sequence positions from position 215 to 2212 of SEQ ID NO: 21; a DNA sequence corresponding to nucleotide sequences 65 to 2965 of SEQ ID NO: 25; a DNA sequence corresponding to nucleotide sequence positions from 159 to 737 of SEQ ID NO: 29; and a DNA sequence corresponding to nucleotide sequence positions from 1435 to 1685 of SEQ ID NO: 33, wherein each of the DNA sequences encodes the protooncoprotein as defined in claim 1.

3. The human protooncogene according to claim 2, wherein the protooncogene has a DNA sequence selected from the group consisting of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29 and SEQ ID NO: 33.

4. A vector comprising each of the protooncogenes as defined in claim 2.

5. A kit for diagnosing cancer and cancer metastasis including each of the protooncoproteins as defined in claim 1.

6. A kit for diagnosing cancer and cancer metastasis including each of the protooncogenes as defined in claim 2.
